(19) United States
(12) Patent Application Publication
(10) Pub. No.: US 2012/0015830 A1
(43) Pub. Date: Jan. 19, 2012

(54) MICRORNA PROFILES FOR EVALUATING MULTIPLE SCLEROSIS

(75) Inventors: DOUGLAS W. BIGWOOD, MADISON, CT (US); ERIC M. EASTMAN, HIGHLAND, MD (US); MICHAEL ELASHOFF, REDWOOD CITY, CA (US)

(73) Assignee: DIOGENIX, INC., GAITHERSBURG, MD (US)

(21) Appl. No.: 13/165,492

(22) Filed: Jun. 21, 2011

Related U.S. Application Data
(60) Provisional application No. 61/356,936, filed on Jun. 21, 2010.
(60) Provisional application No. 61/442,583, filed on Feb. 14, 2011.
(60) Provisional application No. 61/446,324, filed on Feb. 24, 2011.

(51) Int. Cl. C40B 30/00; C07H 21/02; C12Q 1/68

(52) U.S. Cl. 506/7; 435/6.12; 536/24.5; 536/24.33; 436/94; 435/6.1

(57) ABSTRACT

The present invention provides methods, systems, and kits for evaluating multiple sclerosis (MS) in a patient. Particularly, the invention provides convenient miRNA-based tests for evaluating a patient for MS, including for diagnosing MS, for excluding MS as a diagnosis, for determining the presence of disease activity associated with MS, and for monitoring the course of disease or efficacy of treatment for MS.

### PRIORITY

This application claims priority to U.S. Provisional Application No. 61/356,936 filed Jun. 21, 2010, U.S. Provisional Application No. 61/442,583 filed Feb. 14, 2011, and U.S. Provisional Application No. 61/446,324 filed Feb. 24, 2011, each of which is hereby incorporated by reference in its entirety.

### FIELD OF THE INVENTION

The present invention relates to evaluating or discriminating multiple sclerosis (MS) using miRNA profiles, to thereby assist in the diagnosis, prognosis, and/or treatment of MS.

### SEQUENCE LISTING

The contents of the text file submitted electronically herewith are incorporated herein by reference in their entirety: A computer readable format copy of the Sequence Listing (filename: DIOG_{—}003_{—}03US_SeqList_ST25.txt, date recorded: Jun. 17, 2011, file size 20 kilobytes).

### BACKGROUND OF THE INVENTION

Multiple sclerosis (MS) is a disease that affects the central nervous system, and can range from relatively benign to somewhat disabling to devastating. In MS, the myelin surrounding nerve cells is damaged or destroyed, impacting the ability of the nerves to conduct electrical impulses to and from the brain, and leaving scar tissue called sclerosis. These damaged areas are also known as “plaques” or “lesions.”

The first symptoms of MS typically appear between the ages of 20 and 40, and include blurred or double vision, red-green color distortion, or even blindness in one eye. Most MS patients experience muscle weakness in their extremities and difficulty with coordination and balance. In severe cases, MS can produce partial or complete paralysis. Paresthesias (numbness, prickling, or “pins and needles”), speech impediments, tremors, and dizziness are frequent symptoms of MS. Approximately half of MS patients experience cognitive impairments.

Diagnosing MS is complicated, because there is no single test that can confirm the presence of MS. The process of diagnosing MS typically involves criteria from the patient's history, a clinical examination, and one or more laboratory tests, with all three often being necessary to rule out other possible causes for symptoms and/or to gather facts sufficient for a diagnosis of MS.

Magnetic resonance imaging (MRI) is a preferred test. An MRI can detect plaques or scarring possibly caused by MS. However, an abnormal MRI does not necessarily indicate MS, as lesions in the brain may be associated with other disorders. Further, spots may also be found in healthy individuals, particularly in healthy older persons. These spots are called UBOs, for unidentified bright objects, and are not related to an ongoing disease process. In addition, a normal MRI does not absolutely rule out the presence MS. About 5% of individuals who are confirmed to have MS on the basis of other criteria, have no brain lesions detectable by MRI. These individuals may have lesions in the spinal cord or may have lesions that cannot be detected by MRI.

While a diagnosis of MS might be based on an evaluation of symptoms, signs, and the results of an MRI, additional tests may also be ordered. These include tests of evoked potential, cerebrospinal fluid, and blood. For example, cerebrospinal fluid is sampled by a lumbar puncture, and is tested for levels of immune system proteins and for the presence of an antibody staining pattern called “oligoclonal bands.” Oligoclonal bands indicate an immune response within the central nervous system and are found in the spinal fluid of 90-95% of individuals with MS. However, oligoclonal bands are also associated with diseases other than MS, and therefore the presence of oligoclonal bands alone is not definitive of MS.

There is likewise no definitive blood test for MS, but blood tests can exclude other possible causes for various neurologic symptoms, such as Lyme disease, collagen-vascular diseases, rare hereditary disorders, and AIDS.

Diagnosing MS generally requires: (1) objective evidence of at least two areas of myelin loss, or demyelinating lesions, “separated in time and space” (lesions occurring in different places within the brain, spinal cord, or optic nerve-at different points in time); and (2) all other diseases that can cause similar neurologic symptoms have been objectively excluded. Until (1) and (2) are satisfied, a physician does not make a definite diagnosis of MS.

Depending on the clinical problems present when an individual sees a physician, one or more of the tests described above might be performed. Sometimes tests are performed several times over a period of months to help gather the necessary information. A definite MS diagnosis must satisfy the McDonald criteria, named for the distinguished neurologist W. Ian McDonald who sparked society-supported efforts to make the diagnostic process for MS faster and more precise.

There are a few distinct clinical courses for MS, referred to as relapsing-remitting MS, secondary-progressive MS, progressive-relapsing MS, and primary progressive MS. Relapsing-remitting MS is characterized by clearly-defined, acute attacks (relapses), usually with full or partial recovery, and no disease progression between attacks. Secondary-progressive MS is initially relapsing-remitting but then becomes continuously progressive at a variable rate, with or without occasional relapses along the way. The disease-modifying medications are thought to provide benefit for those who continue to have relapses. Primary progressive MS may be characterized by disease progression from the beginning with few or no periods of remission. Progressive-relapsing MS is characterized by disease progression from the beginning, but with clear, acute relapses along the way.

There are several options available for treating individuals diagnosed with MS. Beta-interferon (Avonex, Betaseron, Rebif) has been approved to treat MS. Interferons are also made by the body, mainly to combat viral infections. Interferons have been shown to decrease the worsening or relapse of MS, however disease progression remains unaffected and the side effects of interferons are poorly tolerated. Glatiramer acetate (Copaxone) is a mixture of amino acids that has been shown to decrease the relapse rates of MS by 30%, and appears to also have a positive effect on the overall level of disability. Glatiramer acetate is better tolerated than the interferons and has fewer side effects. Glatiramer acts by binding to major histocompatibility complex class II molecules and competing with MBP and other myelin proteins for such binding and presentation to T cells. Natalizumab (Tysabri) is a monoclonal antibody that binds to alpha-4-integrin on white blood cells and interferes with their movement from the bloodstream into the brain and spinal cord.

An object of the present invention is to provide a convenient diagnostic test for a more objective, definitive, and rapid diagnosis of MS. Another object of the invention is to provide a diagnostic test for monitoring MS progression, adequacy of treatment, and/or response to treatment.

Other objects of the invention will be apparent from the following description of the invention.

### SUMMARY OF THE INVENTION

The present invention provides methods, systems, and kits for evaluating multiple sclerosis (MS) in a patient. Particularly, the invention provides convenient miRNA-based tests for evaluating a patient for MS, including for diagnosing MS, for excluding MS as a diagnosis, for evaluating disease activity indicative of or associated with MS, and for monitoring the course of disease or efficacy of treatment for MS.

In one aspect, the invention provides a method for evaluating a patient for MS. For example, the patient may be suspected of having MS, either due to the presence of demyelinating lesions consistent with MS, or the presence of symptoms of a neurologic and/or immunologic disorder consistent with MS. Alternatively, the patient be undergoing treatment for MS. In this aspect, the method comprises preparing a miRNA profile from a biofluid sample of the patient, and determining the presence or absence of a miRNA signature indicative of MS. The miRNA profile comprises the level or abundance of a plurality of miRNAs of Table 1, Table 2, Table 3, Table 4, or Table 5. In particular, the profile may comprise the level of a plurality of miRNAs that are discriminatory for MS over healthy individuals, such as miRNAs described in Tables 2 and 3. Alternatively, or in addition, the profile may comprise the level of a plurality of miRNAs that are discriminatory for MS over other conditions, such as miRNAs listed in Tables 4 and 5. Table 1 discloses all miRNAs listed in Tables 2 through 5.

The sample, which may be obtained pre- or post- treatment for MS, is a biofluid sample, such as a serum or plasma sample (e.g., a cell-free blood sample), or in other embodiments, a whole blood or peripheral blood mononuclear cell (PBMC) sample. In still other embodiments, the sample is urine, saliva, or cerebrospinal fluid. In certain embodiments, the sample is a serum sample, which may be collected with the use of a serum separator tube, “red-top” tube or clot activator tube. RNA may be subsequently isolated from the serum for miRNA profiling. The miRNA profile is determined by an amplication and/or hybridization-based assay, including, for example, Real-Time PCR (e.g., TaqMan). Other exemplary detection platforms, including direct miRNA capture and miRNA hybridization arrays, are described herein.

The miRNA profile represents the absolute or relative level or abundance of miRNAs present in the sample, and comprises levels for a plurality of miRNAs of Table 1, 2, 3, 4 or 5. In various embodiments, the miRNA profile comprises the level of at least 4, 6, 8, 10, 20, 25, or more miRNAs of Table 1, 2, 3, 4 or 5. In certain embodiments, the miRNA profile is prepared with the use of a custom kit or array, e.g., to allow particularly for the profiling of miRNAs associated with MS. Such profiling may involve determining the level of 150 miRNAs or less, or in other embodiments 100 miRNAs or less, 75 miRNAs or less, 50 miRNAs or less, 25 miRNAs or less, or 10 miRNAs or less, and including miRNAs of Tables 1, 2, 3, 4, or 5.

In particular embodiments, the miRNA profile comprises the level of expression for at least one, two, three, four, five, or each of, hsa-miR-125a-3p, hsa-miR-132, hsa-miR-148b, hsa-miR-181a, hsa-miR-210, hsa-miR-29c, hsa-miR-31, hsa-miR-331-3p, hsa-miR-335, hsa-miR-375, and hsa-miR-483-5p. These miRNAs, which are listed in Table 1, are further listed in the signatures exemplified in both Tables 2 and 3, and which are shown herein to discriminate MS patients from healthy controls.

In some embodiments, the miRNA profile comprises the level of expression for at least one or two of, or each of, hsa-miR-29c, hsa-miR-483-5p, hsa-miR-210, hsa-miR-193b, hsa-miR-186, hsa-miR-192, hsa-miR-132, and hsa-miR-181a. These miRNAs (among others), whose levels are associated with MS, are listed in the signature of Table 2, and shown herein to discriminate MS patients and healthy controls (see FIGS. 1-8).

In particular embodiments, the miRNA profile comprises the level of expression for at least hsa-miR-29c, hsa-miR-483-5p, hsa-miR-210, hsa-miR-132, and hsa-miR-181a. These miRNAs, whose levels are associated with MS, are listed in the signatures exemplified in both Tables 2 and 3.

The miRNA profile is evaluated for the presence or absence of a miRNA signature indicative of MS. The presence or absence of the signature may be determined by any suitable algorithm, which may involve determining whether the miRNA levels are above or below threshold levels that are indicative of MS. In some embodiments, the threshold miRNA levels are set to include about the top or bottom 10% of expression levels as determined in a suitable population of MS patients and healthy controls. Alternatively, the algorithm may involve classifying a sample based upon Mean and/or Median miRNA levels in MS patients versus a non-MS population (e.g., a population of healthy controls or population of patients with diseases other than MS).

The invention thereby provides a predictor for the presence and/or absence of MS, or in some embodiments, the stage and/or progression of MS, the presence of absence of disease activity indicative of or associated with MS, or the efficacy of treatment for MS. The method in certain embodiments provides a positive predictive value for the presence of MS of at least 80%, or at least 85%, or at least 90%, or at least 94%.

In another aspect, the invention provides a method for preparing a miRNA profile indicative of the presence or absence of multiple sclerosis (MS) or indicative of MS disease activity. The method comprises preparing a miRNA profile from a biofluid, such as a serum or plasma sample (e.g., a cell-free blood sample), of a patient suspected of having MS. The miRNA profile comprises the level of 150 miRNAs or less, and includes at least 2 miRNAs of Table 1, 2, 3, 4, or 5. In certain embodiments, the miRNA profile comprises the level of at least 4, or at least 6, or at least 8, or at least 10, or at least 20, or at least 25 miRNAs of Table 1, 2, 3, 4, or 5. The miRNA profile may be prepared with the use of a custom kit or array, e.g., to allow particularly for the profiling of miRNAs associated with MS. Such profiling may involve determining the level of 100 miRNAs or less, 75 miRNAs or less, 50 miRNAs or less, 25 miRNAs or less, or 10 miRNAs or less, including miRNAs of Table 1, 2, 3,4 or 5.

In particular embodiments, the miRNA profile comprises the level of expression for at least one, two, three, four, five, or each of, hsa-miR-125a-3p, hsa-miR-132, hsa-miR-148b, hsa-miR-181a, hsa-miR-210, hsa-miR-29c, hsa-miR-31, hsa-miR-331-3p, hsa-miR-335, hsa-miR-375, and hsa-miR-483-5p. In some embodiments, the miRNA profile comprises the level of expression for at least one or two of, or each of, hsa-miR-29c, hsa-miR-483-5p, hsa-miR-210, hsa-miR-193b, hsa-miR-186, hsa-miR-192, hsa-miR-132, and hsa-miR-181a. In particular embodiments, the miRNA profile comprises the level of expression for at least hsa-miR-29c, hsa-miR-483-5p, hsa-miR-210, hsa-miR-132, and hsa-miR-181a. The miRNA profile may be determined by a variety of detection platforms as described herein, including Real-Time PCR (e.g., TaqMan).

In another aspect, the invention provides a kit or test for preparing a miRNA profile indicative of the presence or absence of MS, or the presence or absence of disease activity associated with MS, and/or for evaluating a patient sample for MS. The kit or test may be configured for a variety of miRNA detection platforms as described herein.

Other aspects and embodiments of the invention will be apparent to the skilled artisan in view of the following detailed description.

### DESCRIPTION OF THE FIGURES

FIG. 1 shows normalized expression levels of miR-29c in the serum of MS patients and healthy controls, as determined by RT-PCR. A normalized expression level within the highest 10% of observed expression levels is indicative of MS.

FIG. 2 shows normalized expression levels of miR-483-5p in the serum of MS patients and healthy controls, as determined by RT-PCR. A normalized expression level within the highest 10% of observed expression levels is indicative of MS.

FIG. 3 shows normalized expression levels of miR-210 in serum of MS patients and healthy controls, as determined by RT-PCR. A normalized expression level within the highest 10% of observed expression levels is indicative of MS.

FIG. 4 shows normalized expression levels of miR-193b in serum of MS patients and healthy controls, as determined by RT-PCR. A normalized expression level within the highest 10% of observed expression levels is indicative of MS.

FIG. 5 shows normalized expression levels of miR-186 in serum of MS patients and healthy controls, as determined by RT-PCR. A normalized expression level within the highest 10% of observed expression levels is indicative of MS.

FIG. 6 shows normalized expression levels of miR-192 in serum of MS patients and healthy controls, as determined by RT-PCR. A normalized expression level within the highest 10% of observed expression levels is indicative of MS.

FIG. 7 shows normalized expression levels of miR-132 in serum of MS patients and healthy controls, as determined by RT-PCR. A normalized expression level within the highest 10% of observed expression levels is indicative of MS.

FIG. 8 shows normalized expression levels of miR-181a in serum of MS patients and healthy controls, as determined by RT-PCR. A normalized expression level within the lowest 10% of observed expression levels is indicative of MS.

### DETAILED DESCRIPTION OF THE INVENTION

The present invention provides methods, systems, and kits for evaluating multiple sclerosis (MS). The invention provides convenient miRNA-based tests for evaluating MS in patients. Such patients may be known to have MS, may be suspected of having MS on the basis of one or more MS-like symptoms or results from one or more MS-related clinical exams, or may be beginning or undergoing treatment for MS. In the various aspects of the invention, the invention aids in diagnosing MS, or excluding MS as a diagnosis, determining MS disease activity, or monitoring the progression of MS or a demyelinating disease consistent with MS, or determining efficacy of an MS treatment.

MicroRNAs (miRNAs) are small (22nt on average) non-coding RNA molecules that have been identified in plants, animals, and other organisms. miRNAs are involved in the post-transcriptional regulation (e.g., silencing) of gene expression, and act by binding to complementary sequences in target messenger RNA transcripts (mRNAs). The human genome may encode over 1000 different miRNAs. miRNAs are associated with fundamental biological processes, including hematopoietic differentiation, cell cycle regulation, metabolism, cardiovascular biology, and immune function. miRNAs can also be associated with the presence and/or progression of disease. See, Calin et al., A microRNA signature associated with prognosis and progression in chronic lymphocytic leukemia, N. Engl. J. Med. 353:1793-1801 (2005); Barbarotto et al., MicroRNAs and cancer: profile, profile, profile. Int. J. Cancer 122:969-977 (2008). The present invention is based, in-part, on the association of miRNA levels with MS.

Under the standard nomenclature system, miRNAs with nearly identical sequences, e.g., bar one or two nucleotides, are annotated with a lower case letter. For example, miR-123a is closely related by sequence to miR-123b. The prefix “hsa” indicates the human (Homo sapiens) sequence. When two mature microRNAs originate from opposite arms of the same pre-miRNA, they are identified with a −3p or −5p suffix.

Methods For Evaluating MS

In some embodiments, the patient is suspected of having MS. For example, the patient may be suspected of having MS on the basis of neurologic and/or immunologic symptoms consistent with MS, e.g., after an initial physician's exam. The patient may, in some embodiments, be positive for the presence of oligoclonal bands. In these or other embodiments, the patient may have CNS lesions characteristic of MS, which are observable on an MRI. In certain embodiments, the patient has been diagnosed as having MS. The patient may not be undergoing treatment for MS, but in some embodiments, the patient is already undergoing treatment, such as treatment with Beta-interferon, Glatiramer acetate, and Natalizumab.

Thus, the patient may have one or more presumptive signs of a multiple sclerosis. Presumptive signs of multiple sclerosis include for example, altered sensory, motor, visual or proprioceptive system with at least one of numbness or weakness in one or more limbs, often occurring on one side of the body at a time or the lower half of the body, partial or complete loss of vision, frequently in one eye at a time and often with pain during eye movement, double vision or blurring of vision, tingling or pain in numb areas of the body, electric-shock sensations that occur with certain head movements, tremor, lack of coordination or unsteady gait, fatigue, dizziness, muscle stiffness or spasticity, slurred speech, paralysis, problems with bladder, bowel or sexual function, and mental changes such as forgetfulness or difficulties with concentration, relative to medical standards.

The sample, which may be obtained pre- or post- treatment for MS, is a biofluid sample, such as a cell-free blood sample (e.g., serum, plasma, or fraction thereof), or in other embodiments, is a whole blood sample or PBMC sample. In still other embodiments, the sample is urine, saliva, or cerebrospinal fluid collected from the patient. miRNAs have been detected, not only in association with blood cells, including PBMCs and platelets, but also in biofluid samples including serum, plasma, urine, and saliva. Hunter et al., Detection of microRNA Expression in Human Peripheral Blood Microvesicles, PloS One Vol. 3, Issue 11 (November 2008); Mitchell et al., Circulating microRNAs as stable blood-based markers for cancer detection, PNAS 105(30):10513-10518 (2008); and Hanke et al., A robust methodology to study urine microRNA as tumor marker: microRNA-126 and microRNA-182 are related to urinary bladder cancer, UrolOnc (Apr. 17, 2009). Thus, in some embodiments, the sample is a serum sample, and which is conveniently and reproducibly collected using, e.g., a serum separator tube or comparable device (e.g., red-top tube or clot activator tube). Various products for serum or plasma collection are well known and commercially available.

In some embodiments, RNA is extracted from the sample prior to miRNA processing for detection. RNA may be purified using a variety of standard procedures as described, for example, in RNA Methodologies, A laboratory guide for isolation and characterization, 2nd edition, 1998, Robert E. Farrell, Jr., Ed., Academic Press. In addition, there are various processes as well as products commercially available for isolation of small molecular weight RNAs, including mirVANA™ Paris miRNA Isolation Kit (Ambion), miRNeasy™ kits (Qiagen), MagMAX™ kits (Life Technologies), and Pure Link™ kits (Life Technologies). For example, small molecular weight RNAs may be isolated by organic extraction followed by purification on a glass fiber filter. Alternative methods for isolating miRNAs include hybridization to magnetic beads.

Alternatively, miRNA processing for detection (e.g., cDNA synthesis) may be conducted in the biofluid sample, that is, without an RNA extraction step.

The miRNA profile (and/or miRNA signature) is generated from samples using any of various techniques known in the art for quantifying miRNA levels, and exemplary detection platforms are described elsewhere herein. Briefly, such methods include, without limitation, polymerase-based assays, such as quantitative RNA-PCR, incuding real-time PCR (e.g., Taqman™), microarray or bead-based hybridization platforms, flap-endonuclease-based assays (e.g., Invader™), as well as direct miRNA capture. For example, miRNA expression can be quantified in a two-step polymerase chain reaction (PCR) process including reverse transcriptase PCR, followed by quantitative real-time PCR. For larger profiles, miRNAs can be hybridized to microarrays, beads, slides or chips. Various commercial products are available for quantifying miRNA levels including the TaqMan Low Density microRNA Array card (TLDA card) (Applied Biosystems Inc.).

In various embodiments, the miRNA profile comprises the absolute or relative level (or abundance) of miRNAs present in the sample, and includes the levels for a plurality of miRNAs of Table 1, or subsets disclosed in Tables 2 to 5. Table 1 includes all miRNAs disclosed in Tables 2 to 5.

TABLE 1

| miR | SEQ ID NO: |
|---|---|
| hsa-miR-181a | 1 |
| hsa-miR-331-3p | 2 |
| hsa-miR-29c | 3 |
| hsa-miR-335 | 4 |
| hsa-miR-483-5p | 5 |
| hsa-miR-193b | 6 |
| hsa-miR-30b | 7 |
| hsa-miR-132 | 8 |
| hsa-miR-181c | 9 |
| hsa-miR-122 | 10 |
| hsa-miR-28-5p | 11 |
| hsa-miR-191 | 12 |
| hsa-miR-30c | 13 |
| hsa-miR-199a-3p | 14 |
| hsa-miR-29a | 15 |
| hsa-miR-145 | 16 |
| hsa-miR-99b | 17 |
| hsa-miR-328 | 18 |
| hsa-miR-26a | 19 |
| hsa-miR-340 | 20 |
| hsa-miR-511 | 21 |
| hsa-miR-192 | 22 |
| hsa-miR-330-3p | 23 |
| hsa-miR-34a | 24 |
| hsa-miR-148a | 25 |
| hsa-miR-140-5p | 26 |
| hsa-miR-210 | 27 |
| hsa-miR-186 | 28 |
| hsa-miR-885-5p | 29 |
| hsa-miR-642 | 30 |
| hsa-miR-212 | 31 |
| hsa-miR-345 | 32 |
| hsa-miR-148b | 33 |
| hsa-miR-375 | 34 |
| hsa-miR-660 | 35 |
| hsa-miR-21 | 36 |
| hsa-miR-532-5p | 37 |
| hsa-miR-202 | 38 |
| hsa-miR-185 | 39 |
| hsa-miR-197 | 40 |
| hsa-miR-628-5p | 41 |
| hsa-miR-31 | 42 |
| hsa-miR-671-3p | 43 |
| hsa-miR-25 | 44 |
| hsa-miR-576-3p | 45 |
| hsa-miR-95 | 46 |
| hsa-miR-218 | 47 |
| hsa-miR-320 | 48 |
| hsa-miR-346 | 49 |
| hsa-miR-518d-3p | 50 |
| hsa-miR-200c | 51 |
| hsa-miR-493 | 52 |
| hsa-miR-379 | 53 |
| hsa-miR-548c-3p | 54 |
| hsa-miR-339-5p | 55 |
| hsa-miR-125a-3p | 56 |
| hsa-miR-874 | 57 |
| hsa-miR-143 | 58 |
| hsa-miR-15a | 59 |
| hsa-miR-193a-3p | 60 |
| hsa-miR-196b | 61 |
| hsa-miR-220b | 62 |
| hsa-miR-32 | 63 |
| hsa-miR-455-5p | 64 |
| hsa-miR-486-5p | 65 |
| hsa-miR-487a | 66 |
| hsa-miR-496 | 67 |
| hsa-miR-502-5p | 68 |
| hsa-miR-523 | 69 |
| hsa-miR-548d-3p | 70 |
| hsa-miR-99a | 71 |
| hsa-miR-106a | 72 |
| hsa-miR-17 | 73 |
| hsa-miR-20b | 74 |
| hsa-miR-484 | 75 |
| hsa-miR-19a | 76 |
| hsa-miR-16 | 77 |
| hsa-miR-342-3p | 78 |
| hsa-miR-502-3p | 79 |
| hsa-miR-19b | 80 |
| hsa-miR-140-3p | 81 |
| hsa-miR-20a | 82 |
| hsa-miR-93 | 83 |
| hsa-miR-18b | 84 |
| hsa-miR-451 | 85 |
| hsa-miR-590-5p | 86 |
| hsa-miR-487b | 87 |
| hsa-miR-139-5p | 88 |
| hsa-miR-146b-5p | 89 |
| hsa-miR-195 | 90 |
| hsa-miR-139-3p | 91 |
| hsa-miR-425 | 92 |
| hsa-miR-625 | 93 |
| hsa-miR-598 | 94 |
| hsa-miR-874 | 95 |
| hsa-miR-376a | 96 |
| hsa-miR-106b | 97 |
| hsa-miR-524-5p | 98 |
| hsa-miR-15a | 99 |
| hsa-miR-363 | 100 |
| hsa-miR-183 | 101 |
| hsa-miR-500 | 102 |
| hsa-miR-589 | 103 |
| hsa-miR-146b-3p | 104 |
| hsa-miR-223 | 105 |
| hsa-miR-324-3p | 106 |
| hsa-miR-301a | 107 |
| hsa-miR-26b | 108 |
| hsa-miR-323-3p | 109 |
| hsa-let-7b | 110 |
| hsa-miR-27a | 111 |
| hsa-miR-429 | 112 |
| hsa-miR-190 | 113 |
| hsa-miR-485-3p | 114 |
| hsa-miR-885-3p | 115 |
| hsa-miR-22 | 116 |
| hsa-miR-374a | 117 |
| hsa-miR-340 | 118 |
| hsa-miR-329 | 119 |
| hsa-miR-455-3p | 120 |
| hsa-miR-345 | 121 |
| hsa-miR-518f | 122 |
| hsa-miR-520b | 123 |
| hsa-miR-561 | 124 |
| hsa-miR-512-3p | 125 |
| hsa-miR-654-3p | 126 |
| hsa-miR-296-5p | 127 |
| hsa-miR-361-5p | 128 |
| hsa-miR-150 | 129 |
| hsa-miR-224 | 130 |
| hsa-miR-182 | 131 |

The nucleotide sequences of the miRNAs listed in Table 1 are known, are hereby incorporated by reference, and are disclosed in the accompanying Sequence Listing. In various embodiments, the miRNA profile comprises the level of at least about 4, 6, 8, 10, 20, 25, 30, 50, 75, 100, or 125 (or all) miRNAs of Table 1. miRNA levels may be expressed in accordance with the selected detection assay. For example, where Real-Time PCR (RT-PCR) is conducted, miRNA levels may be expressed in terms of cycle threshold (CT) values. The Ct or threshold cycle value is the cycle number at which the signal (e.g., fluorescence) generated within a reaction crosses the signal threshold, for example, a fluorescent signal significantly above the background fluorescence. At the threshold cycle, a detectable amount of amplicon product has been generated during the early exponential phase of the reaction. The threshold cycle is inversely proportional to the original relative expression level of the miRNA of interest. The CT values may be normalized as described herein. Alternatively, the profile may be determined by microarray analysis, and the miRNA levels expressed by relative hybridization signal intensity, as normalized for variables such as background, sample processing, and hybridization efficiency.

In various embodiments, the miRNA profile comprises the level of at least about 4, 6, 8, 10, 20, 25 or more miRNAs of Table 2, which as shown herein, may be used to discriminate MS patients (e.g., relapsing remitting MS patients) from healthy controls (see Example 1). miRNA levels may be expressed in accordance with the selected detection assay as described herein.

In other embodiments, the miRNA profile comprises the level of at least about 4, 6, 8, 10, 20, or 25 miRNAs of Table 3, which as shown herein, may be used to discriminate MS patients (e.g., relapsing remitting MS patients) from healthy controls (see Example 2). miRNA levels may be expressed in accordance with the selected detection assay.

The miRNA profile may be prepared with the use of a custom kit or array, e.g., to allow particularly for the profiling of miRNAs associated with MS. Such profiling may involve determining the level of 150 miRNAs or less, or in other embodiments 100 miRNAs or less, 75 miRNAs or less, 50 miRNAs or less, 25 miRNAs or less, including 4, 6, 8, 10, 20, or more miRNAs of Table 1. In some embodiments, at least 25%, or at least 50%, or at least 75% of the miRNAs of the profile are listed in Table 1, 2, or 3.

In certain embodiments, the miRNA profile includes the level of miRNAs associated with, or that discriminates, a non-MS autoimmune disorder, inflammatory disorder, or infectious disease to better discriminate disease states having overlapping symptoms, such as myelitis, systemic lupus erythematosus, Sjögren's syndrome, vasculitis, sarcoidosis, Behget's disease, Lyme disease, syphilis, progressive multifocal leukoencephalopathy, herpes zoster, lysosomal disorder, adrenoleukodystrophy, and CNS lymphoma. For example, the miRNA profile may further discriminate RRMS from one or more of Acute Disseminated Encephalomyelitis, Neuromyelitis Optica, Optic Neuritis, Primary Progressive MS, Psoriasis, Rheumatoid Arthritis, Systemic Lupus Erythematosus, Secondary Progressive Multiple Sclerosis, and Tansverse Myelitis. Exemplary discriminatory miRNA signatures in accordance with these embodiments are shown in Tables 4 and 5. For example, in certain embodiments the miRNA profile includes a determination of the level of (including the level of at least 3 or 5 of) hsa-miR-484, hsa-miR-185, hsa-miR-328, hsa-miR-186, hsa-miR-25, hsa-miR-320, hsa-miR-192, which are shown herein for discriminating MS from healthy controls (Tables 2 and 3), and MS from other diseases (Table 4).

In particular embodiments, the miRNA profile comprises the level of expression for at least one, two, three, four, five, or each of, hsa-miR-125a-3p, hsa-miR-132, hsa-miR-148b, hsa-miR-181a, hsa-miR-210, hsa-miR-29c, hsa-miR-31, hsa-miR-331-3p, hsa-miR-335, hsa-miR-375, and hsa-miR-483-5p. These miRNAs, which are listed in Table 1, are further listed in the signatures exemplified in both Tables 2 and 3, and which are shown herein to discriminate MS patients from healthy controls.

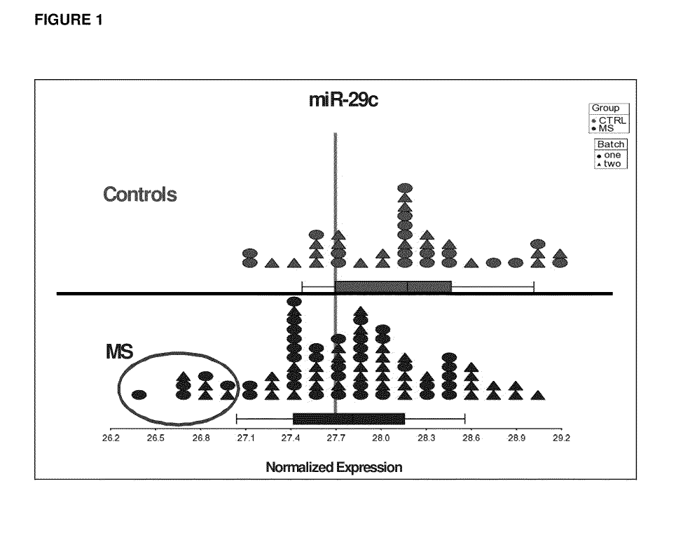

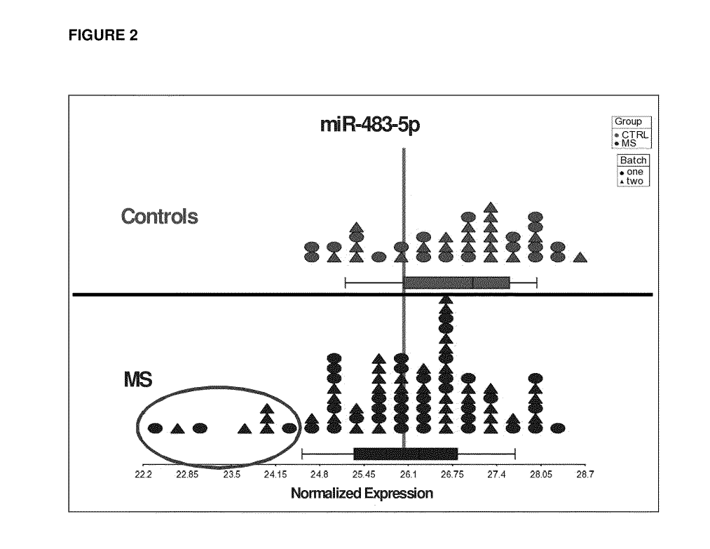

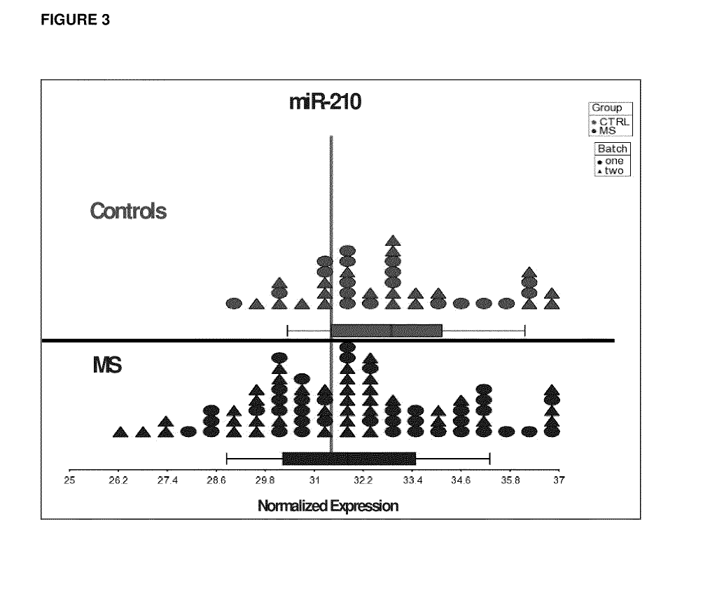

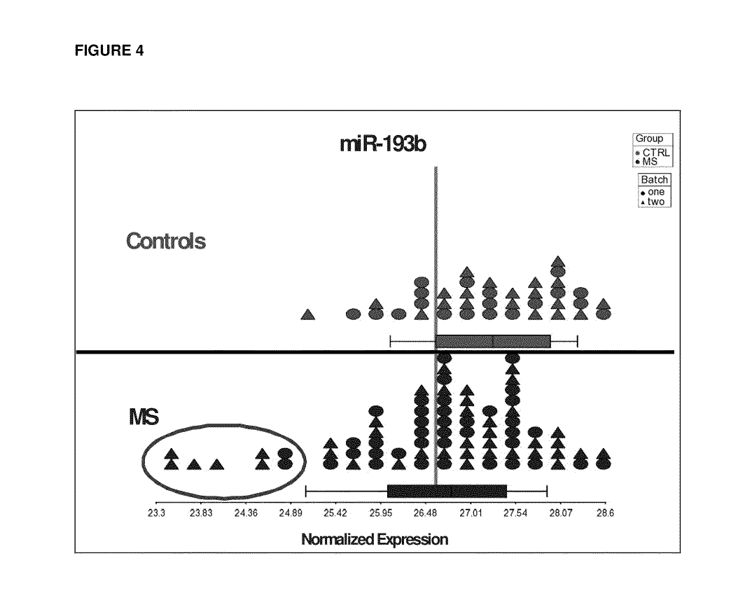

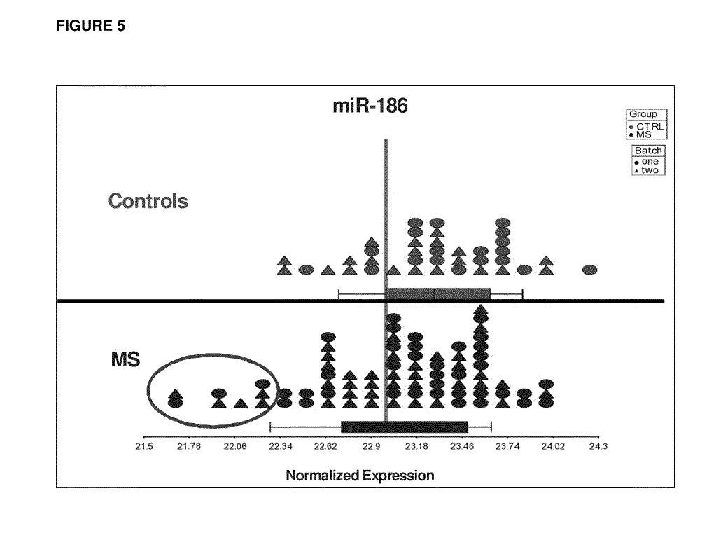

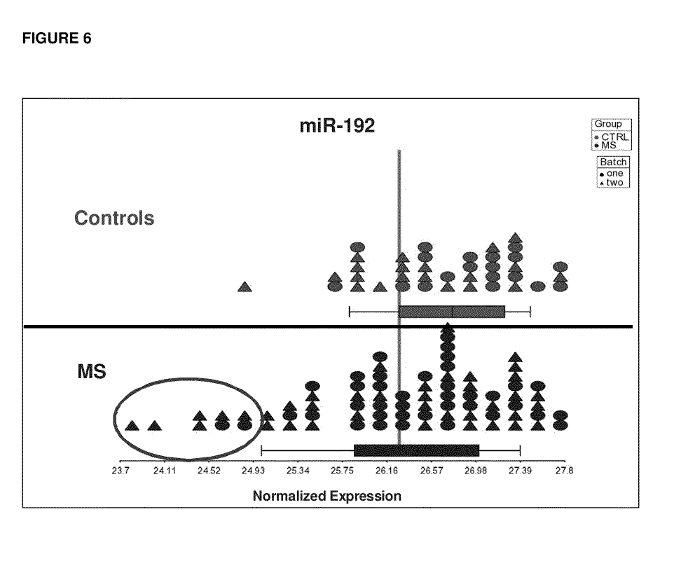

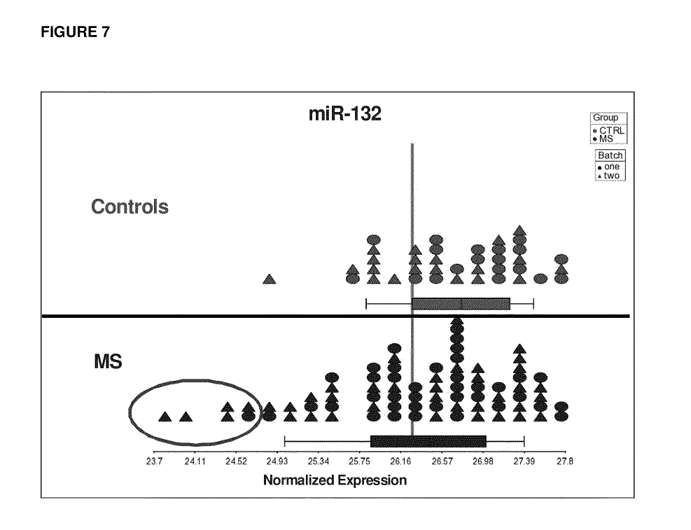

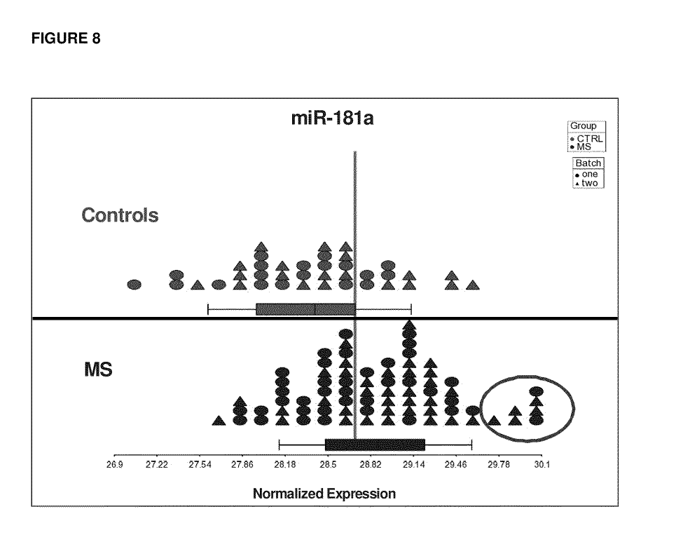

In some embodiments, the miRNA profile comprises the level of expression for at least one or two of, or each of, hsa-miR-29c, hsa-miR-483-5p, hsa-miR-210, hsa-miR-193b, hsa-miR-186, hsa-miR-192, hsa-miR-132, and hsa-miR-181a. These miRNAs (among others), whose levels are associated with MS, are listed in the signature of Table 2, and shown herein to discriminate MS patients and healthy controls. See FIGS. 1-8.

In particular embodiments, the miRNA profile comprises the level of expression for at least hsa-miR-29c, hsa-miR-483-5p, hsa-miR-210, hsa-miR-132, and hsa-miR-181a. These miRNAs, whose levels are associated with MS, are listed in the signatures exemplified in both Tables 2 and 3.

The method may further comprise determining the presence of at least one control RNA to normalize expression levels across samples. For example, the normalization control may be one or more exogenously added RNA(s) or miRNA(s) that are not naturally present in the sample. The normalization control in certain embodiments comprises an Arabidopsis miRNA, such as ath-miR-159a, and/or one or more human miRNAs not expressed in the sample undergoing analysis (e.g., serum). Alternatively or in addition, other methods of normalizing expression levels may be employed, such as normalizing based upon the Mean or Median level of all miRNAs on a given assay run. Methods for normalizing miRNA expression levels are described in Benes and Castoldi, Expression profiling of microRNA using real-time quantitative PCR, how to use it and what is available, Methods 50:244-249 (January 2010); Mestdagh et al., A novel and universal method for microRNA RT-qPCR data normalization, Genome Biology 10:R64 (Jun. 16, 2009).

The miRNA profile is evaluated for the presence or absence of a miRNA signature indicative of MS, or indicative of MS disease activity. The presence or absence of the signature may be determined by any suitable algorithm, which may involve determining the presence of threshold miRNA levels that are indicative of MS. In some embodiments, the threshold miRNA levels are set to include (as indicative of MS) about the top or bottom 10% (e.g., top and bottom 5% to 15%) of expression levels as determined in a suitable population of MS patients and healthy controls. In such embodiments, the use of increasing numbers of miRNAs from Table 1, 2, 3, 4 or 5 may increase predictive value.

Alternatively or in addition, the algorithm may involve classifying a sample between MS and non-MS groups. For example, samples may be classified on the basis of threshold values as described, or based upon Mean and/or Median miRNA levels in MS patients versus a non-MS population (e.g., a population of healthy controls or population of patients with diseases other than MS). Various classification schemes are known for classifying samples between two or more classes or groups, and these include, without limitation: Principal Components Analysis, Naïve Bayes, Support Vector Machines, Nearest Neighbors, Decision Trees, Logistic, Artificial Neural Networks, Penalized Logistic Regression, and Rule-based schemes. In addition, the predictions from multiple models can be combined to generate an overall prediction. For example, a “majority rules” prediction may be generated from the outputs of a Naïve Bayes model, a Support Vector Machine model, and a Nearest Neighbor model.

Thus, a classification algorithm or “class predictor” may be constructed to classify samples. The process for preparing a suitable class predictor is reviewed in R. Simon, Diagnostic and prognostic prediction using gene expression profiles in high-dimensional microarray data, British Journal of Cancer (2003) 89, 1599-1604, which review is hereby incorporated by reference in its entirety.

MS and non-MS signatures for classifying samples may be assembled from miRNA expression data, which may be stored in a database and correlated to patient profiles. MS and non-MS signatures may be selected for a particular patient by, for example, age, race, gender, and/or clinical manifestations of MS. The MS signatures may represent a particular clinical course of MS, such as relapsing-remitting MS, secondary-progressive MS, progessive-relapsing MS, and primary progressive MS. Such additional demographic criteria, such as age, race, gender, MS treatment, and clinical manifestation and course of MS, may be used as factors in the classifier algorithm.

The invention thereby provides an accurate predictor for the presence and/or absence of MS, or the presence or absence of MS disease activity, and in some embodiments provides a positive predictive value of at least 85%, at least 90%, or at least 94%. In various embodiments, the method according to this aspect of the invention distinguishes a MS-afflicted patient (e.g., a relapsing-remitting MS patient) from a non-MS afflicted patient with at least about 50%, 75%, 80%, 85%, 90%, 95%, 97%, 98%, 99% or greater accuracy. In this respect, the method according to this aspect may lend additional or alternative predictive value over standard clinical methods of diagnosing MS, such as for example, absence or presence of lesions on an MRI, testing positive or negative for oligoclonal bands, or the absence or presence of other signs and symptoms of MS such as blurred vision, fatigue, and/or loss of balance.

In certain embodiments where the patient is determined to have MS (e.g., RR MS) on the basis of a gene signature described herein, the patient may be subsequently treated for MS, such as by administration of a treatment indicated for MS. Such treatments include immunomodulating therapy (e.g., beta-inteferon), glatiramer acetate, or Natalizumab. Where MS is excluded as a diagnosis, the patient is not administered an MS treatment.

Methods for Preparing miRNA Profiles

In another aspect, the invention provides a method for preparing a miRNA profile indicative of the presence or absence of MS, or the presence or absence of MS disease activity. The method comprises preparing a miRNA profile from a biofluid sample, such as a serum or plasma sample (or fraction thereof) of a patient suspected of having MS. The miRNA profile includes the level of expression of 150 miRNAs or less, and includes at least 2 miRNAs of Table 1, 2, 3, 4 or 5. In certain embodiments, the miRNA profile comprises the level of at least 4, or at least 6, or at least 8, or at least 10, or at least 20, or at least 25 miRNAs of Table 1, 2, 3, 4, or 5. The miRNA profile may be prepared with the use of a custom kit or array, e.g., to allow particularly for the profiling of miRNAs associated with MS. In certain embodiments, the miRNA profile includes the level of miRNAs associated with at least one non-MS autoimmune disorder, inflammatory disorder or infectious disease, to better discriminate disease states having overlapping symptoms, such as systemic lupus erythematosus, Sjögren's syndrome, vasculitis, sarcoidosis, Behget's disease, Lyme disease, syphilis, progressive multifocal leukoencephalopathy, herpes zoster, lysosomal disorder, adrenoleukodystrophy, and CNS lymphoma. For example, the miRNA profile may further discriminate RRMS from one or more of Acute Disseminated Encephalomyelitis, Neuromyelitis Optica, Optic Neuritis, Primary Progressive MS, Psoriasis, Rheumatoid Arthritis, Systemic Lupus Erythematosus, Secondary Progressive Multiple Sclerosis, and Tansverse Myelitis. Exemplary discriminatory miRNA signatures in accordance with these embodiments are shown in Tables 4 and 5.

Such profiling may involve determining the expression level of 150 miRNAs or less, or in other embodiments 100 miRNAs or less, 75 miRNAs or less, 50 miRNAs or less, 25 miRNAs or less, or 10 miRNAs or less, including miRNAs from Table 1, 2, 3, 4, or 5. In some embodiments, at least 25%, or at least 50%, or at least 75% of the miRNAs of the profile are listed in Table 1, 2, 3, 4, or 5.

In certain embodiments the miRNA profile includes a determination of the level of (including the level of at least 3 or 5 of) hsa-miR-484, hsa-miR-185, hsa-miR-328, hsa-miR-186, hsa-miR-25, hsa-miR-320, hsa-miR-192, which are shown herein for discriminating MS from healthy controls (Tables 2 and 3), and MS from other diseases (Tables 4).

In particular embodiments, the miRNA profile comprises the level of expression for at least one, two, three, four, five, or each of, hsa-miR-125a-3p, hsa-miR-132, hsa-miR-148b, hsa-miR-181a, hsa-miR-210, hsa-miR-29c, hsa-miR-31, hsa-miR-331-3p, hsa-miR-335, hsa-miR-375, and hsa-miR-483-5p. In some embodiments, the miRNA profile comprises the level of expression for at least one or two of, or each of, hsa-miR-29c, hsa-miR-483-5p, hsa-miR-210, hsa-miR-193b, hsa-miR-186, hsa-miR-192, hsa-miR-132, and hsa-miR-181a. In particular embodiments, the miRNA profile comprises the level of expression for at least hsa-miR-29c, hsa-miR-483-5p, hsa-miR-210, hsa-miR-132, and hsa-miR-181a.

The miRNA expression profile is determined by an amplification and/or hybridization-based assay, including, for example, Real-Time PCR (e.g., TaqMan). Suitable detection formats are described in more detail below. miRNA levels may be expressed in accordance with the selected detection assay. For example, where real time PCR is conducted, miRNA levels may be expressed in terms of cycle threshold (CT) values. CT values may be normalized as described herein. Alternatively, the profile may be determined by microarray analysis, and the miRNA levels expressed by relative hybridization signal intensity, as normalized for variables such as background, sample processing, and hybridization efficiency.

The method may further comprise determining the presence of at least one control RNA to normalize expression levels across samples, e.g., with an exogenously added RNA or miRNA as described (e.g., an Arabidopsis miRNA, such as ath-miR-159a, or human miRNA not expressed in the sample undergoing analysis). Alternatively or in addition, other methods of normalizing expression levels may be employed in this aspect of the invention, such as normalizing based upon the Mean or Median level of all miRNAs on a given assay run. Methods for normalizing miRNA expression levels are described in Benes and Castoldi, Expression profiling of microRNA using real-time quantitative PCR, how to use it and what is available, Methods 50:244-249 (January 2010); A novel and universal method for microRNA RT-qPCR data normalization, Genome Biology 10:R64 (Jun. 16, 2009), which are hereby incorporated by reference in their entirety.

Assay Formats

miRNA profiles and miRNA signatures may be prepared according to any suitable method for measuring miRNA levels. That is, the profiles and signatures may be prepared using any quantitative or semi-quantitative method for determining miRNA levels in samples. Such methods include polymerase-based assays, such as Real-Time PCR (e.g., Taqman™), hybridization-based assays, for example using microarrays, nucleic acid sequence based amplification (NASBA), flap endonuclease-based assays, as well as direct RNA capture with branched DNA (QuantiGene™), Hybrid Capture™ (Digene), or nCounter™ miRNA detection (nanostring). The assay format, in addition to determining the miRNA levels will also allow for the control of, inter alia, intrinsic signal intensity variation. Such controls may include, for example, controls for background signal intensity and/or sample processing, and/or hybridization efficiency, as well as other desirable controls for quantifying miRNA levels across samples (e.g., collectively referred to as “normalization controls”). Exemplary assay formats for determining miRNA levels, and thus for preparing miRNA profiles and obtaining data for training MS signatures are described in this section.

The invention may employ reverse transcription PCR and real-time PCR. The application of fluorescence techniques to RT-PCR combined with suitable instrumentation has led to quantitative RT-PCR methods that combine amplification, detection and quantification in a closed system. Two commonly used quantitative RT-PCR techniques are the TaqMan RT-PCR assay (ABI, Foster City, USA) and the Lightcycler assay (Roche, USA). Commercial RT-PCR products for determining miRNA levels are commercially available, and include the TaqMan Low Density miRNA Array card (Applied Biosystems).

The TaqMan detection assays offer certain advantages. First, the methodology makes possible the handling of large numbers of samples efficiently and without cross-contamination and is therefore adaptable for robotic sampling. As a result, large numbers of test samples can be processed in a very short period of time using the TaqMan assay. Another advantage of the TaqMan system is the potential for multiplexing. Since different fluorescent reporter dyes can be used to construct probes, the expression of multiple miRNAs associated with MS could be assayed in the same PCR reaction, thereby reducing the labor costs that would be incurred if each of the tests were performed individually.

Expression profiling of miRNAs using real time quantitative PCR is also described in Benes and Castoldi, Expression profiling of microRNA using real-time quantitative PCR, how to use it and what is available, Methods 50:244-249 (2010); and Chen et al., Reproducibility of quantitative RT-PCR array in miRNA expression profiling and comparison with microarray analysis, BMC Genomics 10:407 (Aug. 28, 2009), each of which is hereby incorporated by reference in its entirety. Briefly, miRNAs present in the sample are converted to cDNA using miRNA-specific primers (either stem-loop or linear miRNA specific primers having a universal 5′ sequence), or by tailing or ligating the miRNAs with a common sequence for priming (e.g., using E. coli poly(A) polymerase or T4 ligase). Amplification of the cDNA may then be quantified in real time, for example, by detecting the signal from a fluorescent reporting molecule, where the signal intensity correlates with the level of DNA at each amplification cycle. Fluorescent technologies include SYBR Green (I or II), which is a DNA-intercalating dye, and TaqMan probes. TaqMan probes have fluorescent and quenching moieties within close proximity, but with the 5′→3′ exonuclease activity of Taq polymerase during amplification, the fluorescent and quencher-containing nucleotides are hydrolyzed and no longer maintained at close proximity by the probe, thereby resulting in fluorescence. In certain embodiments, the cDNA is pre-amplified (e.g., with about 5 to about 15 PCR cycles), prior to real time detection with RT-PCR.

Alternatively, the assay format may employ the methodologies described in Direct Multiplexed Measurement of Gene Expression with Color-Coded Probe Pairs, Nature Biotechnology (Mar. 7, 2008), which describes the nCounter™ Analysis System (nanoString Technologies). This system captures and counts individual RNA transcripts by a molecular bar-coding technology, and is commercialized by Nanostring.

In other embodiments, the invention employs detection and quantification of RNA levels in real-time using nucleic acid sequence based amplification (NASBA) combined with molecular beacon detection molecules. NASBA is described for example, in Compton J., Nucleic acid sequence-based amplification, Nature 1991;350(6313):91-2. NASBA is a singe-step isothermal RNA-specific amplification method. Generally, the method involves the following steps: RNA template is provided to a reaction mixture, where the first primer attaches to its complementary site at the 3′ end of the template; reverse transcriptase synthesizes the opposite, complementary DNA strand; RNAse H destroys the RNA template (RNAse H only destroys RNA in RNA-DNA hybrids, but not single-stranded RNA); the second primer attaches to the 3′ end of the DNA strand, and reverse transcriptase synthesizes the second strand of DNA; and T7 RNA polymerase binds double-stranded DNA and produces a complementary RNA strand which can be used again in step 1, such that the reaction is cyclic.

In yet other embodiments, the assay format is a flap endonuclease-based format, such as the Invader™ assay (Third Wave Technologies). In the case of using the invader method, an invader probe containing a sequence specific to the region 3′ to a target site, and a primary probe containing a sequence specific to the region 5′ to the target site of a template and an unrelated flap sequence, are prepared. Cleavase is then allowed to act in the presence of these probes, the target molecule, as well as a FRET probe containing a sequence complementary to the flap sequence and an auto-complementary sequence that is labeled with both a fluorescent dye and a quencher. When the primary probe hybridizes with the template, the 3′ end of the invader probe penetrates the target site, and this structure is cleaved by the Cleavase resulting in dissociation of the flap. The flap binds to the FRET probe and the fluorescent dye portion is cleaved by the Cleavase resulting in emission of fluorescence.

In yet other embodiments, the assay format employs direct RNA capture with branched DNA (QuantiGene™, Panomics) or Hybrid Capture™ (Digene).

The design of appropriate primers and probes (e.g., TaqMan probes) for reverse transcribing, amplifying, or hybridizing to a particular target miRNA, and as configured for any appropriate nucleic acid detection assay, is well known.

The use of RT-PCR and microarray approaches for determining miRNA levels is described in Chen et al., Reproducibility of quantitative RT-PCR array in miRNA expression profiling and comparison with microarray analysis, BMC Genomics 10:407 (2009), which is hereby incorporated by reference.

Computer Systems

In another aspect, the invention is a computer system that contains a database, on a computer-readable medium, of miRNA expression values determined in an MS patient population and one or more non-MS patient population. These miRNA expression values are determined in biofluid samples, such as serum or plasma or fraction thereof, or in other embodiments, whole blood cell samples, white blood cell samples (e.g., PBMC samples), urine samples, or cerebrospinal fluid samples, and for miRNAs of Table 1, 2, 3, 4, or 5. The database may include, for each miRNA, Mean and/or Median MS and Mean and/or Median Control (e.g., non-MS or healthy) expression levels, as well as various statistical measures, including measures of value dispersion (e.g., Standard Variation), fold change (e.g., between control and MS populations), and statistical significance (statistical association with MS). The database in some embodiments includes threshold expression levels that are indicative of MS for each miRNA associated with MS.

The MS patient population may include patients being treated with Beta-interferon, Glatiramer acetate, and/or Natalizumab, and such treatment and other clinical information may be included in the database such that an appropriate miRNA expression signature may be trained for use with the diagnostic methods of the invention. Generally, signatures may be trained based upon parameters to be selected and input by a user, with these parameters including one or more of age, race, gender, MS treatment, and clinical manifestation and course of MS.

In certain embodiments, the database contains Mean and/or Median miRNA expression values (e.g., expressed as CT threshold or other quantification of expression level) for at least about 5, 8, 10, 20, 40, 50, or all miRNAs of Table 1, 2, 3, 4, or 5. In some embodiments, the database may contain Mean and/or Median miRNA expression levels for more than about 100 miRNAs, or more than about 300 miRNAs, or more than about 400 miRNAs, including those of Table 1. For RT-PCR-based assays, miRNA expression levels may be expressed in terms of CT or change in CT between MS and control groups.

The computer system of the invention may be programmed to classify (e.g., in response to user inputs) a miRNA profile as a non-MS profile or an MS profile, based upon the miRNA expression levels stored and/or generated from the database. For example, the computer system may be programmed to perform any of the known classification schemes for classifying gene expression profiles. Various classification schemes are known for classifying samples, and these include, without limitation: Principal Components Analysis, Naïve Bayes, Support Vector Machines, Nearest Neighbors, Decision Trees, Logistic, Artificial Neural Networks, Penalized Logistic Regression, and Rule-based schemes. The computer system may employ a classification algorithm or “class predictor” as described in R. Simon, Diagnostic and prognostic prediction using gene expression profiles in high-dimensional microarray data, British Journal of Cancer (2003) 89, 1599-1604, which is hereby incorporated by reference in its entirety.

The computer system may further comprise a display, for presenting and/or displaying a result, such as a signature assembled from the database, or the result of a comparison (or classification) between input miRNA expression values and an MS signature. Such results may further be provided in a tangible form (e.g., as a printed report).

The computer system of the invention may further comprise relational databases containing information pertaining to, for instance, the miRNAs of Table 1. For example, the database may contain information associated with a given miRNA, such as descriptive information about the underlying biology and/or pathology of a miRNA and its potential association with disease. Methods for the configuration and construction of databases and computer-readable media to which such databases are saved are widely available, for instance, see U.S. Pat. No. 5,953,727, which is hereby incorporated by reference in its entirety.

The computer system of the invention may be linked to an outside or external database (e.g., on the world wide web) such as GenBank (ncbi.nlm.nih.gov/entrez.index.html) and Sanger website for miRNAs (mirbase.org). In certain embodiments, the external database is GenBank and the associated databases maintained by the National Center for Biotechnology Information (NCBI) (ncbi.nlm.nih.gov), including PubMed.

Diagnostic Kits and Tests

The invention further provides a kit or test for preparing miRNA profiles as described herein. Such miRNA profiles comprise the absolute or relative level (or abundance) of miRNAs present in a sample, and include the levels for a plurality of miRNAs of Table 1, 2, 3, 4, or 5. In various embodiments, the kit is configured to determine the level of at least about 4, 6, 8, 10, 20, 25, 30, 50, or more miRNAs of Table 1, 2, 3, 4, or 5.

The kit may be a custom test or array, e.g., to allow particularly for the profiling of miRNAs associated with MS as described. For example, the kit may comprise probes and/or primers specific for the detection of 150 miRNAs or less, or in other embodiments 100 miRNAs or less, 75 miRNAs or less, 50 miRNAs or less, 25 miRNAs or less, including 4, 6, 8, 10, 20, 25, or more miRNAs of Table 1, 2, 3, 4, or 5.

The test or kit may be configured for a detection system described herein, including RT-PCR (e.g., TaqMan). For example, the kit or test may comprise miRNA-specific primers and/or TaqMan probes for 4, 6, 8, 10, 20, 25 or more miRNAs of Table 1, 2, 3, 4, or 5. Alternatively, the kit may comprise miRNA-specific primers for the miRNAs of Table 1, 2, 3, 4, or 5 and a reagent for detecting / quantifying amplified miRNA, such as SYBR Green dye (I or II). Such kits may further include reagents or tools for miRNA isolation from samples, cDNA preparation (e.g., reverse transcriptase), and PCR amplification (e.g., Taq polymerase).

The primers and/or probes may be designed to detect gene expression levels in accordance with any assay format, including those described herein under the heading “Assay Format.” Exemplary assay formats include polymerase-based assays, such as RT-PCR, TaqMan™, hybridization-based assays, for example using DNA microarrays or other solid support, nucleic acid sequence based amplification (NASBA), flap endonuclease-based assays.

The kit or test may further comprise one or more normalization controls. For example, the normalization control may be an exogenously added RNA or miRNA that is not naturally present in the sample. The normalization control in certain embodiments is an Arabidopsis miRNA, such as ath-miR-159a, or one or more human miRNAs that are not expressed in the sample undergoing analysis (e.g., serum). In such embodiments, the test may further provide miRNA-specific primers for reverse transcribing and/or amplifying the normalization control(s), and a TaqMan probe specific therefore.

The design of miRNA-specific primers (e.g., with a Tm in the range of about 50° C. to about 65° C.) is described in Benes and Castoldi, Expression profiling of microRNA using real-time quantitative PCR, how to use it and what is available, Methods 50:244-249 (2010), which is hereby incorporated by reference in its entirety. The miRNA nucleotide sequences, for designing miRNA-specific primers, are known and are disclosed in the accompanying Sequence Listing.

Without further description, it is believed that one of ordinary skill in the art can, using the preceding description and the following illustrative examples, make and use the present invention.

### EXAMPLES

### Example 1

Inclusion and Exclusion Criteria for Relapsing Remitting MS Patients

Relapsing-remitting (RR) MS patients who meet all of the following inclusion criteria during the initial visit were eligible for enrollment in the study:
(1) Diagnosis of a RRMS as defined by 2005 revised McDonald criteria;
(2) Patients aged 18-65 years of age, inclusive;
(3) Patient is able to provide informed consent.

RRMS patients who meet any of the following exclusion criteria during the visit were not be eligible for enrollment in the study:
(1) A manifestation of MS other than RRMS;
(2) A history of chronic disease of the immune system other than MS or history of a known immunodeficiency syndrome;
(3) A known diagnosis of diabetes mellitus Type 1;
(4) Active systemic bacterial, viral or fungal infections, or diagnosis of AIDS, Hepatitis B, Hepatitis C infection defined as a positive HIV antibody, Hepatitis B surface antigen or Hepatitis C antibody tests, respectively;
(5) Have received total lymphoid irradiation or bone marrow transplantation;
(6) Have been treated with: plasma exchange within last 30 days; lymphacytapheresis within last 30 days; corticosteroids or adrenocorticotropic hormones (ACTH) within 6 months prior to the first visit; IFN, glatiramer acetate, azathiprine, methotrexate, mycophenolate, IVIG, natalizumab in the last 12 months prior to the first visit; Rituximab, cladribine, cyclophosphamide or mitoxantrone at any time;
(7) Current pregnancy.

The inclusion and exclusion criteria for healthy controls were as follows:
(1) Age 18-50,
(2) Not diagnosed with MS or any other inflammatory or autoimmune disease,
(3) No steroids of any kind within last 6 months.

This example includes 79 serum samples from patients diagnosed with MS and 40 healthy controls.

Serum Collection

Blood was collected in Serum Separator Tubes (BD, 8-9 ml of blood each). Blood was allowed to clot for at least 30 minutes and the tubes were centrifuged according to the manufacturer's recommendations within 2 hours after blood collection. Serum was carefully removed from the tube and 0.5 mL aliquots were transferred to barcode-labeled plastic cryovials and frozen.

Serum microRNA Profiling

Individual serum aliquots were processed using the TaqMan Low Density Array (“TLDA card”) platform (Life Technologies—Applied Biosystems) to produce miRNA expression profiles. There are two human TLDA cards, A and B, that cover a total of 667 unique human miRNAs. This example uses the A card, which includes TaqMan assays for 377 individual human miRNAs and 4 control miRNAs (381 total assays). One of the control assays is for a non-human miRNA, ath-miR-159a, which was used to control for variable RNA recovery during the isolation of miRNA from individual serum samples. To simplify sample processing, pools of RT and PCR primers specific for the individual miRNA on each TLDA card are available. “Megaplex” RT and PreAmp primer pools for the TLDA A card contain all the primers required to amplify all 381 targets. The Megaplex RT pool is used to convert miRNA targets to cDNA and the Megaplex PreAmp primer pools are used to amplify the DNA targets prior to TaqMan analysis. The “preamplification” step increases sensitivity of the assay and allows for the detection of miRNAs present at copy numbers too low to be detected using standard TaqMan assays.

Circulating RNA was isolated from 200 uL of serum using a modified RNA isolation protocol based on the miNana Paris miRNA Isolation kit (Life Technologies-Ambion). A fixed concentration of synthetic ath-miR-159a oligonucleotide was spiked into each serum sample after addition of the 2× Denaturing Solution provided in the miNana kit. RNA was converted to cDNA using Megaplex RT primer pools for the TLDA A card (Life Technologies-Applied Biosystems) and amplified prior to TaqMan analysis using Megaplex PreAmp primer pools for the TLDA A card and 14 cycles of PCR. The resulting amplified DNA was then applied to TLDA A cards for TaqMan analysis.

The signature of Table 2 employs 57 miRs from Table 1. Table 2 includes Median and Mean expression values for the control and MS groups. The “Cycle Threshold” (Ct) data was produced using DataAssist 2.0 (Applied Biosystems) using miR-ath-159a as the Selected Control. The Medians and Means were produced using Partek Genomics Suite 6.5 (build 6.10.0412—Partek Inc.).

| miR | FIG. | CTRL - Mean | CTRL - Median | MS - Mean | MS - Median |
|---|---|---|---|---|---|
| hsa-miR-181a | 8 | 28.47 | 28.19 | 28.91 | 28.97 |
| (SEQ ID NO: 1) | | | | | |
| hsa-miR-331-3p | | 25.75 | 25.42 | 26.01 | 25.98 |
| (SEQ ID NO: 2) | | | | | |
| hsa-miR-29c | 1 | 28.25 | 28.07 | 27.85 | 27.83 |
| (SEQ ID NO: 3) | | | | | |
| hsa-miR-335 | | 27.16 | 27.07 | 27.45 | 27.28 |
| (SEQ ID NO: 4) | | | | | |
| hsa-miR-483-5p | 2 | 26.78 | 26.78 | 26.09 | 26.19 |
| (SEQ ID NO: 5) | | | | | |
| hsa-miR-193b | 4 | 27.26 | 27.07 | 26.66 | 26.9 |
| (SEQ ID NO: 6) | | | | | |
| hsa-miR-30b | | 23.7 | 23.3 | 23.87 | 23.72 |
| (SEQ ID NO: 7) | | | | | |
| hsa-miR-132 | 7 | 27.49 | 26.57 | 26.64 | 26.44 |
| (SEQ ID NO: 8) | | | | | |
| hsa-miR-181c | | 33.88 | 33.68 | 34.67 | 34.36 |
| (SEQ ID NO: 9) | | | | | |
| hsa-miR-122 | | 24.67 | 24.66 | 23.96 | 24.35 |
| (SEQ ID NO: 10) | | | | | |
| hsa-miR-28-5p | | 28.39 | 27.79 | 28.45 | 28.26 |
| (SEQ ID NO: 11) | | | | | |
| hsa-miR-191 | | 20.82 | 20.42 | 20.97 | 20.85 |
| (SEQ ID NO: 12) | | | | | |
| hsa-miR-30c | | 22.94 | 22.67 | 23.12 | 22.96 |
| (SEQ ID NO: 13) | | | | | |
| hsa-miR-199a-3p | | 25.27 | 24.93 | 25.46 | 25.46 |
| (SEQ ID NO: 14) | | | | | |
| hsa-miR-29a | | 25.58 | 25.54 | 25.25 | 25.19 |
| (SEQ ID NO: 15) | | | | | |
| hsa-miR-145 | | 25.5 | 25.33 | 25.79 | 25.8 |
| (SEQ ID NO: 16) | | | | | |
| hsa-miR-99b | | 29.2 | 28.9 | 29.32 | 29.2 |
| (SEQ ID NO: 17) | | | | | |
| hsa-miR-328 | | 25.18 | 25.15 | 25.39 | 25.42 |
| (SEQ ID NO: 18) | | | | | |
| hsa-miR-26a | | 23.68 | 23.34 | 23.83 | 23.73 |
| (SEQ ID NO: 19) | | | | | |
| hsa-miR-340 | | 27.09 | 26.82 | 27.3 | 27.16 |
| (SEQ ID NO: 20) | | | | | |
| hsa-miR-511 | | 34.47 | 32.84 | 33.09 | 32 |
| (SEQ ID NO: 21) | | | | | |
| hsa-miR-192 | 6 | 26.79 | 26.74 | 26.37 | 26.61 |
| (SEQ ID NO: 22) | | | | | |
| hsa-miR-330-3p | | 32 | 31.87 | 32.2 | 31.91 |
| (SEQ ID NO: 23) | | | | | |
| hsa-miR-34a | | 31.5 | 30.9 | 30.56 | 30.09 |
| (SEQ ID NO: 24) | | | | | |
| hsa-miR-148a | | 27.25 | 27.06 | 26.96 | 26.99 |
| (SEQ ID NO: 25) | | | | | |
| hsa-miR-140-5p | | 24.68 | 24.31 | 24.39 | 24.25 |
| (SEQ ID NO: 26) | | | | | |
| hsa-miR-210 | 3 | 33.24 | 32.73 | 32 | 31.56 |
| (SEQ ID NO: 27) | | | | | |
| hsa-miR-186 | 5 | 23.38 | 22.98 | 23.11 | 23.09 |
| (SEQ ID NO: 28) | | | | | |
| hsa-miR-885-5p | | 26.3 | 26.21 | 25.77 | 25.94 |
| (SEQ ID NO: 29) | | | | | |
| hsa-miR-642 | | 33.47 | 31.77 | 32.18 | 31.65 |
| (SEQ ID NO: 30) | | | | | |
| hsa-miR-212 | | 32.07 | 31.08 | 31.33 | 30.8 |
| (SEQ ID NO: 31) | | | | | |
| hsa-miR-345 | | 26.03 | 25.92 | 25.74 | 25.63 |
| (SEQ ID NO: 32) | | | | | |
| hsa-miR-148b | | 30.32 | 29.65 | 29.84 | 29.78 |
| (SEQ ID NO: 33) | | | | | |
| hsa-miR-375 | | 26.69 | 26.8 | 26.25 | 26.43 |
| (SEQ ID NO: 34) | | | | | |
| hsa-miR-660 | | 26.11 | 25.75 | 25.7 | 25.91 |
| (SEQ ID NO: 35) | | | | | |
| hsa-miR-21 | | 24.07 | 23.78 | 23.8 | 23.87 |
| (SEQ ID NO: 36) | | | | | |
| hsa-miR-532-5p | | 26.48 | 26.07 | 26.18 | 26.25 |
| (SEQ ID NO: 37) | | | | | |
| hsa-miR-202 | | 33.7 | 32.41 | 32.82 | 31.82 |
| (SEQ ID NO: 38) | | | | | |
| hsa-miR-185 | | 26.75 | 26.5 | 26.49 | 26.52 |
| (SEQ ID NO: 39) | | | | | |
| hsa-miR-197 | | 24.74 | 24.35 | 24.84 | 24.73 |
| (SEQ ID NO: 40) | | | | | |
| hsa-miR-628-5p | | 31.57 | 30.46 | 32.16 | 30.92 |
| (SEQ ID NO: 41) | | | | | |
| hsa-miR-31 | | 32.28 | 31.98 | 31.64 | 31.27 |
| (SEQ ID NO: 42) | | | | | |
| hsa-miR-671-3p | | 30.31 | 29.92 | 30.58 | 30.08 |
| (SEQ ID NO: 43) | | | | | |
| hsa-miR-25 | | 24.59 | 24.44 | 24.29 | 24.49 |
| (SEQ ID NO: 44) | | | | | |
| hsa-miR-576-3p | | 32.29 | 31.85 | 31.9 | 31.53 |
| (SEQ ID NO: 45) | | | | | |
| hsa-miR-95 | | 33.47 | 31.84 | 32.73 | 31.59 |
| (SEQ ID NO: 46) | | | | | |
| hsa-miR-218 | | 33.88 | 32.24 | 33.01 | 31.89 |
| (SEQ ID NO: 47) | | | | | |
| hsa-miR-320 | | 21.4 | 21.25 | 21.23 | 21.22 |
| (SEQ ID NO: 48) | | | | | |
| hsa-miR-346 | | 33.91 | 33.57 | 34.36 | 34 |
| (SEQ ID NO: 49) | | | | | |
| hsa-miR-518d-3p | | 33.52 | 33.18 | 33.9 | 33.04 |
| (SEQ ID NO: 50) | | | | | |
| hsa-miR-200c | | 30.75 | 29.46 | 30.54 | 30.13 |
| (SEQ ID NO: 51) | | | | | |
| hsa-miR-493 | | 34.98 | 34.08 | 34.96 | 33.37 |
| (SEQ ID NO: 52) | | | | | |
| hsa-miR-379 | | 34.14 | 32.94 | 34.3 | 32.88 |
| (SEQ ID NO: 53) | | | | | |
| hsa-miR-548c-3p | | 35.65 | 33.89 | 36.55 | 35.61 |
| (SEQ ID NO: 54) | | | | | |
| hsa-miR-339-5p | | 34.99 | 34.9 | 35.61 | 35.76 |
| (SEQ ID NO: 55) | | | | | |
| hsa-miR-125a-3p | | 33.5 | 33.06 | 35.03 | 33.7 |
| (SEQ ID NO: 56) | | | | | |
| hsa-miR-874 | | 36.98 | 36.83 | 35.78 | 35.35 |
| (SEQ ID NO: 57) | | | | | |

Discriminating MS

FIGS. 1-8 illustrate an exemplary model for discriminating MS, using one or more of miR-29c, miR-483-5p, miR-210, miR-193b, miR-186, miR-192, miR-132, and miR-181a. Cutoff values were set to include the top 10% highest (or lowest 10% for miR-181a) expressing samples.

The 8-miR test provides a Positive Predictive Value (PPV) of about 94%, and a Negative Predictive Value (NPV) of about 45%.

### Example 2

A second set of 25 miRNAs from Table 1, which are indicative of RRMS versus healthy controls, was generated using Penalized Logistic Regression. Goeman J. J. (2010). L-1 Penalized Estimation in the Cox Proportional Hazards Model. Biometrical Journal 52 (1) 70-84. The same laboratory procedures described in Example 1 were used for data generation. Data was normalized as follows.
1. Set all values greater than 38 to 38.
2. Compute average pairwise correlation for each sample.
3. Drop samples with average correlation <0.88.
4. Drop microRNAs with 90% of the values=38.
5. Compute sample means (across all remaining microRNAs).
6a. If marker has 30% values=38, normalize marker by subtracting off sample means.
6b. If marker has >30% values=38, make marker a binary indicator of value=38 vs value <38.
7. Scale each microRNA to have a mean of 0 and a variance of 1.

Validation of this miRNA profile was performed on an independent set of data for 73 RRMS patients and 61 Healthy controls. The miRNA signature in this embodiment is shown in Table 3, below.

| miR | CTRL - Mean | CTRL - Median | MS - Mean | MS - Median |
|---|---|---|---|---|
| hsa-miR-125a-3p | −0.349294015 | −0.674033293 | 0.174647008 | −0.0109357 |
| (SEQ ID NO: 56) | | | | |
| hsa-miR-132 | 0.354253361 | −0.08055708 | −0.17712668 | −0.2749867 |
| (SEQ ID NO: 8) | | | | |
| hsa-miR-143 | 0.206644416 | −0.105078477 | −0.103322208 | −0.1526048 |
| (SEQ ID NO: 58) | | | | |
| hsa-miR-148b | 0.280100744 | 0.24400447 | −0.140050372 | −0.1503672 |
| (SEQ ID NO: 33) | | | | |
| hsa-miR-15a | 0.097467553 | 0.154856764 | −0.048733777 | −0.2579503 |
| (SEQ ID NO: 59) | | | | |
| hsa-miR-181a | −0.53610382 | −0.384706206 | 0.26805191 | 0.34451375 |
| (SEQ ID NO: 1) | | | | |
| hsa-miR-193a-3p | 0.175411604 | −0.584705346 | −0.087705802 | −0.5847053 |
| (SEQ ID NO: 60) | | | | |
| hsa-miR-196b | −0.199858088 | −0.458334842 | 0.099929044 | −0.2836464 |
| (SEQ ID NO: 61) | | | | |
| hsa-miR-210 | 0.280380717 | 0.286012541 | −0.140190359 | −0.1862864 |
| (SEQ ID NO: 27) | | | | |
| hsa-miR-220b | −0.25246042 | −0.336613894 | 0.12623021 | −0.3366139 |
| (SEQ ID NO: 62) | | | | |
| hsa-miR-29c | 0.415834798 | 0.349524057 | −0.207917399 | −0.2594697 |
| (SEQ ID NO: 3) | | | | |
| hsa-miR-31 | 0.198327221 | 0.033550612 | −0.09916361 | −0.2275263 |
| (SEQ ID NO: 42) | | | | |
| hsa-miR-32 | −0.311128023 | 0.677160992 | 0.155564012 | 0.67716099 |
| (SEQ ID NO: 63) | | | | |
| hsa-miR-331-3p | −0.376070321 | −0.359891446 | 0.18803516 | 0.16184129 |
| (SEQ ID NO: 2) | | | | |
| hsa-miR-335 | −0.351700935 | −0.315069502 | 0.175850468 | 0.12734129 |
| (SEQ ID NO: 4) | | | | |
| hsa-miR-375 | 0.270944624 | 0.35548277 | −0.135472312 | −0.0241135 |
| (SEQ ID NO: 34) | | | | |
| hsa-miR-455-5p | 0.191655223 | −0.801467295 | −0.095827611 | −0.8014673 |
| (SEQ ID NO: 64) | | | | |
| hsa-miR-483-5p | 0.406656629 | 0.592017987 | −0.203328314 | −0.0374406 |
| (SEQ ID NO: 5) | | | | |
| hsa-miR-486-5p | −0.113082154 | −0.384854507 | 0.056541077 | 0.20748811 |
| (SEQ ID NO: 65) | | | | |
| hsa-miR-487a | −0.095460041 | −0.610944262 | 0.04773002 | −0.6109443 |
| (SEQ ID NO: 66) | | | | |
| hsa-miR-496 | −0.212287647 | −0.424575294 | 0.106143823 | −0.4245753 |
| (SEQ ID NO: 67) | | | | |
| hsa-miR-502-5p | −0.174232021 | −0.801467295 | 0.08711601 | −0.8014673 |
| (SEQ ID NO: 68) | | | | |
| hsa-miR-523 | 0.206181761 | −0.637289079 | −0.10309088 | −0.6372891 |
| (SEQ ID NO: 69) | | | | |
| hsa-miR-548d-3p | 0.283050196 | −0.424575294 | −0.141525098 | −0.4245753 |
| (SEQ ID NO: 70) | | | | |
| hsa-miR-99a | −0.187840703 | −0.307054614 | 0.093920351 | −0.227789 |
| (SEQ ID NO: 71) | | | | |

The Area Under the Curve for the model was 0.67 with a p-value of 0.0002. The Positive Predictive Value was 82.1% and the Negative Predictive Value was 52.8%.

### Example 3

A third set of 32 miRNAs, which are indicative of RRMS versus Other Diseases (OD), using a Mann-Whitney U test. In this example, data—generated as described above for Example 1—from 100 RRMS subject samples were compared against 142 samples from subjects with ODs. The ODs included the following diseases with the number of subjects in parentheses: Acute Disseminated Encephalomyelitis (13), Neuromyelitis Optica (9), Optic Neuritis (7), Primary Progressive Multiple Sclerosis (19), Psoriasis (19), Rheumatoid Arthritis (20), Systemic Lupus Erythematosus (16), Secondary Progressive Multiple Sclerosis (19), and Transverse Myelitis (20). Prior to applying the Mann-Whitney test the data was normalized as follows:
1. The mean of all miRNAs with a Ct value of less than 35 for a subject was subtracted from all values for a subject;
2. The value of all miRNAs with a raw Ct value of greater than or equal to 35 was set to 15.

The miRNAs with a p-value less than 0.05 are listed in the following Table 4.

| | | OD - | RRMS - | RRMS - |
|---|---|---|---|---|
| miRNA | OD - Mean | Median | Mean | Median |
| hsa-miR-106a | −7.58095 | −7.73403 | −7.90631 | −8.07473 |
| hsa-miR-17 | −7.62032 | −7.7613 | −7.92947 | −8.07816 |
| hsa-miR-20b | −3.50414 | −3.70138 | −3.89952 | −3.99325 |
| hsa-miR-486-5p | −2.85675 | −2.93828 | −3.25623 | −3.3542 |
| hsa-miR-484 | −7.25976 | −7.32165 | −7.54271 | −7.66983 |
| hsa-miR-19a | −4.39608 | −4.55232 | −4.74886 | −4.86624 |
| hsa-miR-16 | −8.88844 | −8.98172 | −9.27885 | −9.39767 |
| hsa-miR-342-3p | −4.41134 | −4.54935 | −4.69237 | −4.73649 |
| hsa-miR-502-3p | 5.28648 | 3.92741 | 4.75155 | 3.41539 |
| hsa-miR-19b | −8.5507 | −8.72387 | −8.89588 | −8.89863 |
| hsa-miR-140-3p | 0.11396 | 0.0158244 | −0.0211716 | −0.22828 |
| hsa-miR-185 | −1.64193 | −1.76089 | −1.93871 | −2.03766 |
| hsa-miR-20a | −6.61933 | −6.78547 | −6.98702 | −7.20153 |
| hsa-miR-328 | −2.57904 | −2.66063 | −2.86973 | −3.17332 |
| hsa-miR-93 | −4.41776 | −4.59049 | −4.75683 | −4.82947 |
| hsa-miR-18b | 10.1245 | 15 | 8.18027 | 5.45026 |
| hsa-miR-451 | −6.97958 | −7.05314 | −7.36171 | −7.52758 |
| hsa-miR-186 | −4.36349 | −4.48022 | −4.62779 | −4.65053 |
| hsa-miR-590-5p | −2.65912 | −2.81108 | −2.91934 | −3.04537 |
| hsa-miR-487b | 5.95904 | 3.32151 | 4.0666 | 2.77581 |
| hsa-miR-139-5p | −2.2016 | −2.4959 | −2.5805 | −2.74094 |
| hsa-miR-25 | −4.11323 | −4.15395 | −4.3559 | −4.3797 |
| hsa-miR-146b-5p | −3.13289 | −3.18854 | −3.36177 | −3.47773 |
| hsa-miR-195 | −3.47741 | −3.5734 | −3.78543 | −3.80726 |
| hsa-miR-139-3p | 5.31716 | 3.36498 | 4.55341 | 2.65301 |
| hsa-miR-320 | −6.38188 | −6.38182 | −6.54396 | −6.56656 |
| hsa-miR-425 | −1.75928 | −1.95107 | −2.04437 | −2.13681 |
| hsa-miR-625 | 4.02109 | 3.23038 | 3.76326 | 2.87874 |
| hsa-miR-192 | −2.0911 | −2.21059 | −2.37684 | −2.43579 |
| hsa-miR-598 | 2.28585 | 1.25349 | 1.59088 | 0.820656 |
| hsa-miR-874 | 12.317 | 15 | 10.8327 | 15 |
| hsa-miR-376a | 0.874454 | 0.299911 | 0.0155651 | −0.05615 |

### Example 4

A fourth set of miRNAs differentially expressed among all diseases, including RRMS, are listed in Example 3 using a one-way ANOVA with each disease as a separate group. The same samples and the same method of normalization was used as in Example 3. The miRNAs with p-values less than 0.05 are listed in the following Table 5.

| | Mean | Mean | Mean | Mean | Mean | Mean | Mean | Mean | Mean | Mean |
|---|---|---|---|---|---|---|---|---|---|---|
| miRNA | (ADEM) | (NMO) | (ON) | (PPMS) | (Psoriasis) | (RA) | (RRMS) | (SLE) | (SPMS) | (TM) |
| hsa-miR-106b-4373155 | −4.572 | −4.174 | −4.764 | −4.045 | −5.346 | −3.618 | −4.364 | −3.819 | −4.026 | −4.783 |
| hsa-miR-29a-4395223 | −3.321 | −3.391 | −3.976 | −2.802 | −3.912 | −2.548 | −3.234 | −2.804 | −2.939 | −3.447 |
| hsa-miR-524-5p-4395174 | 15.000 | 15.000 | 12.089 | 15.000 | 15.000 | 15.000 | 15.000 | 15.000 | 15.000 | 15.000 |
| hsa-miR-140-5p-4373374 | −3.766 | −3.623 | −4.281 | −3.256 | −3.941 | −2.654 | −3.537 | −3.065 | −3.121 | −3.622 |
| hsa-miR-186-4395396 | −4.671 | −4.293 | −5.190 | −4.192 | −4.816 | −3.842 | −4.628 | −3.953 | −4.261 | −4.587 |
| hsa-miR-29c-4395171 | −0.874 | −0.433 | −1.301 | −0.270 | −1.206 | 0.233 | −0.681 | −0.127 | −0.375 | −0.954 |
| hsa-miR-15a-4373123 | 7.618 | 7.686 | 7.003 | 10.116 | 5.487 | 13.500 | 9.680 | 9.964 | 9.418 | 6.493 |
| hsa-miR-363-4378090 | 3.305 | 6.547 | 3.155 | 7.306 | 1.564 | 6.345 | 4.555 | 7.707 | 5.098 | 2.911 |
| hsa-miR-18b-4395328 | 10.337 | 9.285 | 6.816 | 11.258 | 7.192 | 13.401 | 8.180 | 12.225 | 10.465 | 7.951 |
| hsa-miR-185-4395382 | −1.871 | −1.486 | −1.832 | −1.332 | −2.407 | −1.083 | −1.939 | −1.125 | −1.696 | −1.985 |
| hsa-miR-532-5p-4380928 | −1.976 | −0.006 | −2.287 | −1.584 | −2.697 | −0.282 | −1.948 | −1.359 | −1.725 | −2.179 |
| hsa-miR-183-4395380 | 11.984 | 10.820 | 12.208 | 11.277 | 8.839 | 14.531 | 12.417 | 13.814 | 12.975 | 10.044 |
| hsa-miR-484-4381032 | −7.573 | −7.159 | −7.654 | −7.144 | −7.438 | −6.875 | −7.543 | −6.862 | −7.398 | −7.477 |
| hsa-miR-106a-4395280 | −7.677 | −7.396 | −7.999 | −7.489 | −8.023 | −7.186 | −7.906 | −7.180 | −7.614 | −7.806 |
| hsa-miR-210-4373089 | 6.213 | 6.039 | 4.815 | 6.222 | 2.212 | 9.198 | 4.595 | 7.225 | 4.930 | 3.803 |
| hsa-miR-19b-4373098 | −8.691 | −8.084 | −8.844 | −8.514 | −9.133 | −8.115 | −8.896 | −8.202 | −8.602 | −8.714 |
| hsa-miR-500-4395539 | 6.906 | 10.311 | 6.879 | 10.257 | 8.121 | 10.915 | 10.388 | 14.267 | 12.799 | 9.572 |
| hsa-miR-487a-4378097 | 11.203 | 13.011 | 9.232 | 13.730 | 13.479 | 15.000 | 13.102 | 14.289 | 10.665 | 11.582 |
| hsa-miR-451-4373360 | −6.818 | −6.237 | −7.172 | −6.834 | −8.031 | −6.442 | −7.362 | −6.650 | −7.046 | −7.228 |
| hsa-miR-17-4395419 | −7.731 | −7.398 | −7.981 | −7.541 | −8.080 | −7.289 | −7.929 | −7.175 | −7.678 | −7.794 |
| hsa-miR-589-4395520 | 11.611 | 12.819 | 9.835 | 14.594 | 11.070 | 14.503 | 13.675 | 13.800 | 12.448 | 13.613 |
| hsa-miR-146b-3p-4395472 | 8.173 | 9.627 | 9.045 | 12.930 | 9.474 | 13.898 | 10.173 | 11.788 | 12.379 | 8.697 |
| hsa-miR-223-4395406 | −12.477 | −12.224 | −13.314 | −11.857 | −12.412 | −11.654 | −12.363 | −11.499 | −11.946 | −12.407 |
| hsa-miR-324-3p-4395272 | 0.296 | 2.070 | −0.171 | 1.063 | −0.615 | 0.853 | 0.123 | 0.714 | 1.212 | 0.089 |
| hsa-miR-487b-4378102 | 4.297 | 7.954 | 3.827 | 4.711 | 5.160 | 9.459 | 4.067 | 6.219 | 5.769 | 5.305 |
| hsa-miR-301a-4373064 | −0.581 | 1.418 | −0.366 | 0.002 | −0.516 | 0.580 | −0.135 | 0.489 | −0.014 | −0.486 |
| hsa-miR-26b-4395167 | −3.422 | −1.235 | −3.959 | −2.979 | −3.725 | −2.528 | −3.414 | −2.686 | −2.991 | −3.521 |
| hsa-miR-16-4373121 | −8.691 | −8.465 | −9.304 | −8.738 | −9.656 | −8.493 | −9.279 | −8.522 | −9.069 | −8.993 |
| hsa-miR-323-3p-4395338 | 2.307 | 1.976 | 2.591 | 2.059 | 2.592 | 2.046 | 2.326 | 1.690 | 2.060 | 2.306 |
| hsa-miR-19a-4373099 | −4.585 | −4.032 | −4.804 | −4.266 | −4.855 | −3.976 | −4.749 | −4.055 | −4.530 | −4.548 |
| hsa-miR-191-4395410 | −7.042 | −6.765 | −7.426 | −6.523 | −6.872 | −6.297 | −6.907 | −6.047 | −6.565 | −6.946 |
| hsa-let-7b-4395446 | −2.590 | −0.633 | −2.712 | −1.253 | −2.944 | −1.323 | −2.481 | −1.645 | −2.075 | −3.014 |
| hsa-miR-93-4373302 | −4.490 | −3.651 | −4.868 | −4.455 | −4.936 | −4.028 | −4.757 | −3.990 | −4.532 | −4.653 |
| hsa-miR-486-5p-4378096 | −2.654 | −1.754 | −2.840 | −2.802 | −3.482 | −2.619 | −3.256 | −2.607 | −3.185 | −3.074 |
| hsa-miR-27a-4373287 | −3.458 | −2.984 | −3.788 | −2.943 | −3.240 | −2.531 | −3.140 | −2.717 | −2.933 | −3.450 |
| hsa-miR-20b-4373263 | −3.522 | −2.942 | −3.832 | −3.513 | −4.002 | −3.148 | −3.900 | −3.136 | −3.579 | −3.728 |
| hsa-miR-429-4373203 | 11.557 | 7.879 | 8.470 | 13.060 | 9.868 | 13.082 | 10.521 | 10.002 | 12.691 | 8.437 |
| hsa-miR-148a-4373130 | −1.622 | 0.439 | −2.014 | −1.159 | −1.579 | −1.559 | −1.628 | −1.212 | −1.588 | −1.631 |
| hsa-miR-190-4373110 | 6.146 | 5.828 | 7.960 | 8.730 | 4.666 | 10.587 | 6.415 | 7.070 | 7.563 | 5.346 |
| hsa-miR-485-3p-4378095 | 0.735 | 4.535 | 0.975 | 1.666 | 3.381 | 1.917 | 1.367 | 1.597 | 1.270 | 1.288 |
| hsa-miR-181c-4373115 | 7.335 | 11.691 | 7.461 | 10.600 | 10.015 | 13.023 | 9.997 | 11.920 | 10.394 | 8.617 |
| hsa-miR-146b-5p-4373178 | −3.226 | −3.083 | −3.561 | −2.979 | −3.709 | −2.870 | −3.362 | −2.851 | −2.840 | −3.310 |
| hsa-miR-885-3p-4395483 | 15.000 | 13.748 | 15.000 | 15.000 | 14.463 | 15.000 | 15.000 | 15.000 | 15.000 | 15.000 |
| hsa-miR-25-4373071 | −3.913 | −3.848 | −4.244 | −3.887 | −4.820 | −3.851 | −4.356 | −3.744 | −4.228 | −4.310 |
| hsa-miR-22-4373079 | 7.873 | 9.782 | 6.913 | 10.657 | 8.049 | 12.864 | 9.186 | 13.118 | 10.215 | 8.452 |
| hsa-miR-34a-4395168 | 2.846 | 4.093 | 3.431 | 4.951 | 1.639 | 6.039 | 3.245 | 2.727 | 6.385 | 2.702 |
| hsa-miR-374a-4373028 | −3.511 | −3.166 | −3.782 | −3.003 | −3.661 | −2.736 | −3.337 | −2.670 | −2.802 | −3.620 |
| hsa-miR-340-4395369 | −1.233 | 0.539 | −1.808 | −0.788 | −0.951 | −0.689 | −1.127 | −0.476 | −0.854 | −1.273 |
| hsa-miR-329-4373191 | 12.051 | 12.981 | 7.961 | 13.017 | 13.087 | 14.544 | 12.802 | 11.899 | 13.929 | 13.680 |
| hsa-miR-455-3p-4395355 | 14.205 | 12.877 | 15.000 | 14.520 | 15.000 | 14.540 | 14.911 | 15.000 | 14.537 | 15.000 |
| hsa-miR-345-4395297 | −2.500 | −2.152 | −2.516 | −2.038 | −2.350 | −1.611 | −2.243 | −1.766 | −2.183 | −2.432 |
| hsa-miR-518f-4395499 | 12.466 | 15.000 | 10.571 | 14.093 | 13.871 | 15.000 | 13.978 | 12.359 | 13.913 | 14.579 |
| hsa-miR-520b-4373252 | 14.328 | 15.000 | 15.000 | 15.000 | 15.000 | 15.000 | 15.000 | 15.000 | 15.000 | 15.000 |
| hsa-miR-561-4380938 | 14.145 | 15.000 | 15.000 | 15.000 | 15.000 | 15.000 | 15.000 | 15.000 | 15.000 | 15.000 |
| hsa-miR-512-3p-4381034 | 14.348 | 10.843 | 12.209 | 12.351 | 13.297 | 13.853 | 13.976 | 14.352 | 15.000 | 12.080 |
| hsa-miR-654-3p-4395350 | 9.350 | 13.826 | 9.070 | 11.343 | 13.919 | 13.471 | 11.565 | 11.057 | 13.223 | 10.510 |
| hsa-miR-296-5p-4373066 | 4.177 | 5.668 | 5.039 | 3.161 | 2.677 | 6.177 | 3.534 | 4.702 | 4.073 | 3.424 |
| hsa-miR-361-5p-4373035 | 1.871 | 3.226 | 1.615 | 2.566 | 2.574 | 5.183 | 2.396 | 4.874 | 1.765 | 1.316 |
| hsa-miR-150-4373127 | −6.258 | −6.388 | −7.038 | −6.458 | −7.537 | −6.675 | −6.856 | −6.548 | −6.335 | −6.421 |
| hsa-miR-224-4395210 | 2.656 | 3.873 | 1.753 | 4.229 | 3.226 | 3.807 | 1.537 | 1.751 | 1.418 | 1.520 |
| hsa-miR-182-4395445 | 9.828 | 7.113 | 7.342 | 7.873 | 5.923 | 10.342 | 7.943 | 10.353 | 10.028 | 6.186 |
| hsa-miR-590-5p-4395176 | −2.924 | −2.353 | −3.484 | −2.443 | −2.984 | −2.320 | −2.919 | −2.329 | −2.730 | −2.769 |

All patents or publications disclosed herein are incorporated by reference in their entireties.

1. A method for evaluating a patient sample for multiple sclerosis (MS), comprising:
preparing a miRNA profile from a biofluid sample collected from the patient, and determining the presence or absence of a miRNA signature indicative of multiple sclerosis, the miRNA profile comprising the level of at least 4 miRNAs of Table 1, 2, 3, 4, or 5.

2. The method of claim 1, wherein the patient is suspected of having MS.

3. The method of claim 2, wherein the patient has demyelinating lesions consistent with MS.

4. The method of claim 2, wherein the patient has symptoms of a neurologic and/or immunologic disorder consistent with MS.

5. The method of claim 1, wherein the miRNA profile is determined prior to treating the patient for MS.

6. The method of claim 1, wherein the miRNA profile is indicative of disease activity associated with MS.

7. The method of claim 1, wherein the miRNA profile is determined in a serum or plasma sample.

8. The method of claim 7, wherein the sample is a serum sample collected with a serum separator tube.

9. The method of claim 1, wherein the miRNA profile comprises the level of at least 5 miRNAs of Table 1, 2, or 3.

10. The method of claim 9, wherein the miRNA profile comprises the level of at least 6 miRNAs of Table 1, 2, or 3.

11. The method of claim 9, wherein the miRNA profile comprises the level of at least 8 miRNAs of Table 1, 2, or 3.

12. (canceled)

13. (canceled)

14. The method of claim 1, wherein the miRNA profile comprises the level for 150 miRNAs or less.

15. The method of claim 14, wherein the miRNA profile comprises the level for 100 miRNAs or less.

16. (canceled)

17. (canceled)

18. (canceled)

19. (canceled)

20. The method of claim 1, wherein the miRNA profile comprises the level for at least one of hsa-miR-125a-3p, hsa-miR-132, hsa-miR-148b, hsa-miR-181a, hsamiR-210, hsa-miR-29c, hsa-miR-31, hsa-miR-331-3p, hsa-miR-335, hsa-miR-375, and hsamiR-483-5p.

21. (canceled)

22. The method of claim 20, wherein the miRNA profile comprises the level for one or more of hsa-miR-29c, hsa-miR-483-5p, hsa-miR-210, hsa-miR-193b, hsa-miR-186, hsamiR-192, hsa-miR-132, and hsa-miR-181a.

23. (canceled)

24. The method of claim 1, further comprising, determining the level of one or more normalization controls in the sample.

25. The method of claim 24, wherein the sample is spiked with the normalization control(s).

26. The method of claim 25, wherein the normalization control is a non-endogenous RNA or miRNA, or a miRNA not expressed in the sample.

27. The method of claim 26, wherein the normalization controls include an Arabidopsis miRNA.

28. The method of claim 27, wherein the normalization control(s) include ath-miR-159a.

29. The method of claim 1, wherein miRNA levels are normalized to the Mean or Median expression level for all miRNAs in the profile.

30. The method of claim 1, wherein miRNA profile is determined by amplication and/or hybridization-based assay.

31. The method of claim 30, wherein the miRNA profile is determined by preparing cDNA, followed by Real Time PCR.

32. The method of claim 31, wherein the Real Time PCR is TaqMan.

33. The method of claim 1, wherein the miRNA signature is an algorithm.

34. The method of claim 33, wherein the miRNA signature involves threshold miRNA expression levels that are indicative of MS.

35. The method of claim 34, wherein the threshold miRNA levels indicative of MS are set to include the top or bottom 5 to 15% of expression levels within a population of MS patients and healthy controls.

36. The method of claim 34, wherein the threshold miRNA levels indicative of MS are above the Mean or Median expression level in samples of a non-MS population.

37. The method of claim 33, wherein the miRNA signature involves Mean or Median miRNA expression levels in MS patients as compared to a non-MS population.

38. The method of claim 1, wherein the method has a positive predictive value of at least 80%.

39. The method of claim 38, wherein the method has a positive predictive value of 90% of greater.

40. (canceled)

41. A method for preparing a miRNA profile indicative of the presence or absence of multiple sclerosis (MS), comprising:
preparing a miRNA profile from a biofluid sample collected from a patient suspected of having MS, the miRNA profile comprising the level of 150 miRNAs or less including at least 4 miRNAs of Table 1, 2, 3, 4, or 5.

42-69. (canceled)

70. A kit for preparing a miRNA profile indicative of the presence or absence of multiple sclerosis (MS), or the presence or absence of disease activity associated with MS, comprising: a miRNA-specific primer for reverse transcribing or amplifying each of 150 miRNAs or less, including at least 4 miRNAs of Table 1, 2, 3, 4, or 5.

71-91. (canceled)


SEQUENCE LISTING (131 sequences)

SEQ ID NO: 1 - RNA, 23 nt, Homo sapiens
aacauucaacgcugucggugagu

SEQ ID NO: 2 - RNA, 21 nt, Homo sapiens
gccccugggccuauccuagaa

SEQ ID NO: 3 - RNA, 22 nt, Homo sapiens
uagcaccauuugaaaucgguua

SEQ ID NO: 4 - RNA, 23 nt, Homo sapiens
ucaagagcaauaacgaaaaaugu

SEQ ID NO: 5 - RNA, 22 nt, Homo sapiens
aagacgggaggaaagaagggag

SEQ ID NO: 6 - RNA, 22 nt, Homo sapiens
aacuggcccucaaagucccgcu

SEQ ID NO: 7 - RNA, 22 nt, Homo sapiens
uguaaacauccuacacucagcu

SEQ ID NO: 8 - RNA, 22 nt, Homo sapiens
uaacagucuacagccauggucg

SEQ ID NO: 9 - RNA, 22 nt, Homo sapiens
aacauucaaccugucggugagu

SEQ ID NO: 10 - RNA, 22 nt, Homo sapiens
uggagugugacaaugguguuug

SEQ ID NO: 11 - RNA, 22 nt, Homo sapiens
aaggagcucacagucuauugag

SEQ ID NO: 12 - RNA, 23 nt, Homo sapiens
caacggaaucccaaaagcagcug

SEQ ID NO: 13 - RNA, 23 nt, Homo sapiens
uguaaacauccuacacucucagc

SEQ ID NO: 14 - RNA, 22 nt, Homo sapiens
acaguagucugcacauugguua

SEQ ID NO: 15 - RNA, 22 nt, Homo sapiens
uagcaccaucugaaaucgguua

SEQ ID NO: 16 - RNA, 23 nt, Homo sapiens
guccaguuuucccaggaaucccu

SEQ ID NO: 17 - RNA, 22 nt, Homo sapiens
cacccguagaaccgaccuugcg

SEQ ID NO: 18 - RNA, 22 nt, Homo sapiens
cuggcccucucugcccuuccgu

SEQ ID NO: 19 - RNA, 22 nt, Homo sapiens
uucaaguaauccaggauaggcu

SEQ ID NO: 20 - RNA, 22 nt, Homo sapiens
uuauaaagcaaugagacugauu

SEQ ID NO: 21 - RNA, 21 nt, Homo sapiens
gugucuuuugcucugcaguca

SEQ ID NO: 22 - RNA, 21 nt, Homo sapiens
cugaccuaugaauugacagcc

SEQ ID NO: 23 - RNA, 23 nt, Homo sapiens
gcaaagcacacggccugcagaga

SEQ ID NO: 24 - RNA, 22 nt, Homo sapiens
uggcagugucuuagcugguugu

SEQ ID NO: 25 - RNA, 22 nt, Homo sapiens
ucagugcacuacagaacuuugu

SEQ ID NO: 26 - RNA, 22 nt, Homo sapiens
cagugguuuuacccuaugguag

SEQ ID NO: 27 - RNA, 22 nt, Homo sapiens
cugugcgugugacagcggcuga

SEQ ID NO: 28 - RNA, 22 nt, Homo sapiens
caaagaauucuccuuuugggcu

SEQ ID NO: 29 - RNA, 22 nt, Homo sapiens
uccauuacacuacccugccucu

SEQ ID NO: 30 - RNA, 22 nt, Homo sapiens
gucccucuccaaaugugucuug

SEQ ID NO: 31 - RNA, 21 nt, Homo sapiens
uaacagucuccagucacggcc

SEQ ID NO: 32 - RNA, 22 nt, Homo sapiens
gcugacuccuaguccagggcuc

SEQ ID NO: 33 - RNA, 22 nt, Homo sapiens
ucagugcaucacagaacuuugu

SEQ ID NO: 34 - RNA, 22 nt, Homo sapiens
uuuguucguucggcucgcguga

SEQ ID NO: 35 - RNA, 22 nt, Homo sapiens
uacccauugcauaucggaguug

SEQ ID NO: 36 - RNA, 22 nt, Homo sapiens
uagcuuaucagacugauguuga

SEQ ID NO: 37 - RNA, 22 nt, Homo sapiens
caugccuugaguguaggaccgu

SEQ ID NO: 38 - RNA, 20 nt, Homo sapiens
agagguauagggcaugggaa

SEQ ID NO: 39 - RNA, 22 nt, Homo sapiens
uggagagaaaggcaguuccuga

SEQ ID NO: 40 - RNA, 22 nt, Homo sapiens
uucaccaccuucuccacccagc

SEQ ID NO: 41 - RNA, 22 nt, Homo sapiens
augcugacauauuuacuagagg

SEQ ID NO: 42 - RNA, 21 nt, Homo sapiens
aggcaagaugcuggcauagcu

SEQ ID NO: 43 - RNA, 21 nt, Homo sapiens
uccgguucucagggcuccacc

SEQ ID NO: 44 - RNA, 22 nt, Homo sapiens
cauugcacuugucucggucuga

SEQ ID NO: 45 - RNA, 22 nt, Homo sapiens
aagauguggaaaaauuggaauc

SEQ ID NO: 46 - RNA, 22 nt, Homo sapiens
uucaacggguauuuauugagca

SEQ ID NO: 47 - RNA, 21 nt, Homo sapiens
uugugcuugaucuaaccaugu

SEQ ID NO: 48 - RNA, 22 nt, Homo sapiens
  misc_feature (1)..(1): n may be a or absent
naaagcuggguugagargrnnn

SEQ ID NO: 49 - RNA, 23 nt, Homo sapiens
ugucugcccgcaugccugccucu

SEQ ID NO: 50 - RNA, 21 nt, Homo sapiens
caaagcgcuucccuuuggagc

SEQ ID NO: 51 - RNA, 23 nt, Homo sapiens
uaauacugccggguaaugaugga

SEQ ID NO: 52 - RNA, 22 nt, Homo sapiens
ugaaggucuacugugugccagg

SEQ ID NO: 53 - RNA, 21 nt, Homo sapiens
ugguagacuauggaacguagg

SEQ ID NO: 54 - RNA, 22 nt, Homo sapiens
caaaaaucucaauuacuuuugc

SEQ ID NO: 55 - RNA, 23 nt, Homo sapiens
ucccuguccuccaggagcucacg

SEQ ID NO: 56 - RNA, 22 nt, Homo sapiens
acaggugagguucuugggagcc

SEQ ID NO: 57 - RNA, 22 nt, Homo sapiens
cugcccuggcccgagggaccga

SEQ ID NO: 58 - RNA, 21 nt, Homo sapiens
ugagaugaagcacuguagcuc

SEQ ID NO: 59 - RNA, 22 nt, Homo sapiens
uagcagcacauaaugguuugug

SEQ ID NO: 60 - RNA, 22 nt, Homo sapiens
aacuggccuacaaagucccagu

SEQ ID NO: 61 - RNA, 22 nt, Homo sapiens
uagguaguuuccuguuguuggg

SEQ ID NO: 62 - RNA, 21 nt, Homo sapiens
ccaccaccgugucugacacuu

SEQ ID NO: 63 - RNA, 22 nt, Homo sapiens
uauugcacauuacuaaguugca

SEQ ID NO: 64 - RNA, 22 nt, Homo sapiens
uaugugccuuuggacuacaucg

SEQ ID NO: 65 - RNA, 22 nt, Homo sapiens
uccuguacugagcugccccgag

SEQ ID NO: 66 - RNA, 22 nt, Homo sapiens
aaucauacagggacauccaguu

SEQ ID NO: 67 - RNA, 22 nt, Homo sapiens
ugaguauuacauggccaaucuc

SEQ ID NO: 68 - RNA, 21 nt, Homo sapiens
auccuugcuaucugggugcua

SEQ ID NO: 69 - RNA, 23 nt, Homo sapiens
gaacgcgcuucccuauagagggu

SEQ ID NO: 70 - RNA, 22 nt, Homo sapiens
caaaaaccacaguuucuuuugc

SEQ ID NO: 71 - RNA, 22 nt, Homo sapiens
aacccguagauccgaucuugug

SEQ ID NO: 72 - RNA, 23 nt, Homo sapiens
aaaagugcuuacagugcagguag

SEQ ID NO: 73 - RNA, 23 nt, Homo sapiens
caaagugcuuacagugcagguag

SEQ ID NO: 74 - RNA, 23 nt, Homo sapiens
caaagugcucauagugcagguag

SEQ ID NO: 75 - RNA, 22 nt, Homo sapiens
ucaggcucaguccccucccgau

SEQ ID NO: 76 - RNA, 23 nt, Homo sapiens
ugugcaaaucuaugcaaaacuga

SEQ ID NO: 77 - RNA, 22 nt, Homo sapiens
uagcagcacguaaauauuggcg

SEQ ID NO: 78 - RNA, 23 nt, Homo sapiens
ucucacacagaaaucgcacccgu

SEQ ID NO: 79 - RNA, 22 nt, Homo sapiens
aaugcaccugggcaaggauuca

SEQ ID NO: 80 - RNA, 23 nt, Homo sapiens
ugugcaaauccaugcaaaacuga

SEQ ID NO: 81 - RNA, 21 nt, Homo sapiens
uaccacaggguagaaccacgg

SEQ ID NO: 82 - RNA, 23 nt, Homo sapiens
uaaagugcuuauagugcagguag

SEQ ID NO: 83 - RNA, 23 nt, Homo sapiens
caaagugcuguucgugcagguag

SEQ ID NO: 84 - RNA, 23 nt, Homo sapiens
uaaggugcaucuagugcaguuag

SEQ ID NO: 85 - RNA, 22 nt, Homo sapiens
aaaccguuaccauuacugaguu

SEQ ID NO: 86 - RNA, 22 nt, Homo sapiens
gagcuuauucauaaaagugcag

SEQ ID NO: 87 - RNA, 22 nt, Homo sapiens
aaucguacagggucauccacuu

SEQ ID NO: 88 - RNA, 22 nt, Homo sapiens
ucuacagugcacgugucuccag

SEQ ID NO: 89 - RNA, 22 nt, Homo sapiens
ugagaacugaauuccauaggcu

SEQ ID NO: 90 - RNA, 21 nt, Homo sapiens
uagcagcacagaaauauuggc

SEQ ID NO: 91 - RNA, 22 nt, Homo sapiens
ggagacgcggcccuguuggagu

SEQ ID NO: 92 - RNA, 23 nt, Homo sapiens
aaugacacgaucacucccguuga

SEQ ID NO: 93 - RNA, 21 nt, Homo sapiens
agggggaaaguucuauagucc

SEQ ID NO: 94 - RNA, 22 nt, Homo sapiens
uacgucaucguugucaucguca

SEQ ID NO: 95 - RNA, 22 nt, Homo sapiens
cugcccuggcccgagggaccga

SEQ ID NO: 96 - RNA, 21 nt, Homo sapiens
aucauagaggaaaauccacgu

SEQ ID NO: 97 - RNA, 21 nt, Homo sapiens
uaaagugcugacagugcagau

SEQ ID NO: 98 - RNA, 22 nt, Homo sapiens
cuacaaagggaagcacuuucuc

SEQ ID NO: 99 - RNA, 22 nt, Homo sapiens
uagcagcacauaaugguuugug

SEQ ID NO: 100 - RNA, 22 nt, Homo sapiens
aauugcacgguauccaucugua

SEQ ID NO: 101 - RNA, 22 nt, Homo sapiens
uauggcacugguagaauucacu

SEQ ID NO: 102 - RNA, 23 nt, Homo sapiens
uaauccuugcuaccugggugaga

SEQ ID NO: 103 - RNA, 22 nt, Homo sapiens
ugagaaccacgucugcucugag

SEQ ID NO: 104 - RNA, 22 nt, Homo sapiens
ugcccuguggacucaguucugg

SEQ ID NO: 105 - RNA, 22 nt, Homo sapiens
ugucaguuugucaaauacccca

SEQ ID NO: 106 - RNA, 20 nt, Homo sapiens
acugccccaggugcugcugg

SEQ ID NO: 107 - RNA, 23 nt, Homo sapiens
cagugcaauaguauugucaaagc

SEQ ID NO: 108 - RNA, 21 nt, Homo sapiens
uucaaguaauucaggauaggu

SEQ ID NO: 109 - RNA, 21 nt, Homo sapiens
cacauuacacggucgaccucu

SEQ ID NO: 110 - RNA, 22 nt, Homo sapiens
ugagguaguagguugugugguu

SEQ ID NO: 111 - RNA, 21 nt, Homo sapiens
uucacaguggcuaaguuccgc

SEQ ID NO: 112 - RNA, 22 nt, Homo sapiens
uaauacugucugguaaaaccgu

SEQ ID NO: 113 - RNA, 22 nt, Homo sapiens
ugauauguuugauauauuaggu

SEQ ID NO: 114 - RNA, 22 nt, Homo sapiens
gucauacacggcucuccucucu

SEQ ID NO: 115 - RNA, 22 nt, Homo sapiens
aggcagcgggguguaguggaua

SEQ ID NO: 116 - RNA, 22 nt, Homo sapiens
aagcugccaguugaagaacugu

SEQ ID NO: 117 - RNA, 22 nt, Homo sapiens
uuauaauacaaccugauaagug

SEQ ID NO: 118 - RNA, 22 nt, Homo sapiens
uuauaaagcaaugagacugauu

SEQ ID NO: 119 - RNA, 22 nt, Homo sapiens
aacacaccugguuaaccucuuu

SEQ ID NO: 120 - RNA, 21 nt, Homo sapiens
gcaguccaugggcauauacac

SEQ ID NO: 121 - RNA, 22 nt, Homo sapiens
gcugacuccuaguccagggcuc

SEQ ID NO: 122 - RNA, 21 nt, Homo sapiens
gaaagcgcuucucuuuagagg

SEQ ID NO: 123 - RNA, 21 nt, Homo sapiens
aaagugcuuccuuuuagaggg

SEQ ID NO: 124 - RNA, 22 nt, Homo sapiens
caaaguuuaagauccuugaagu

SEQ ID NO: 125 - RNA, 22 nt, Homo sapiens
aagugcugucauagcugagguc

SEQ ID NO: 126 - RNA, 22 nt, Homo sapiens
uaugucugcugaccaucaccuu

SEQ ID NO: 127 - RNA, 21 nt, Homo sapiens
agggcccccccucaauccugu

SEQ ID NO: 128 - RNA, 22 nt, Homo sapiens
uuaucagaaucuccagggguac

SEQ ID NO: 129 - RNA, 22 nt, Homo sapiens
ucucccaacccuuguaccagug

SEQ ID NO: 130 - RNA, 21 nt, Homo sapiens
caagucacuagugguuccguu

SEQ ID NO: 131 - RNA, 24 nt, Homo sapiens
uuuggcaaugguagaacucacacu